(19) United States
(12) Patent Application Publication
(10) Pub. No.: US 2023/0331815 A1
(43) Pub. Date: Oct. 19, 2023

(54) METHODS AND COMPOSITIONS FOR TREATING VIRAL INFECTION

(71) Applicant: AVIRUS, INC., Lafayette, CA (US)

(72) Inventors: Jang Hyun HAN, Lafayette, CA (US); William J. RUTTER, San Francisco, CA (US); Mi-Young SEO, Seoul (KR)

(21) Appl. No.: 18/321,610

(22) Filed: May 22, 2023

Related U.S. Application Data
(63) Continuation of application No. PCT/US2021/060732, filed on Nov. 24, 2021.
(60) Provisional application No. 63/118,096, filed on Nov. 25, 2020.

(51) Int. Cl. C07K 14/005; A61K 31/7048; A61K 38/40; A61K 38/16; C07K 14/79; A61P 31/14

(52) U.S. Cl. CPC C07K 14/79; A61K 31/7048; A61K 38/162; A61K 38/40; A61P 31/14; C07K 14/005; C07K 2319/00; C07K 2319/30

(57) ABSTRACT

The disclosure provides methods for treating or preventing coronavirus infection comprising administration of a therapeutically effective amount of lactoferrin or a therapeutically effective amount of a protein fusion comprising recombinant lactoferrin fused to the receptor binding domain of the SARS-CoV-2 spike protein. Also provided are methods for treating or preventing SARS-CoV-2 infection comprising co-administration of a therapeutically effective amount of lactoferrin and a second drug treatment, such as ivermectin. Also provided are methods for treating or preventing SARS-CoV-2 infection comprising co-administration of a therapeutically effective amount of lactoferrin and a second drug treatment, such as ivermectin.

### CROSS REFERENCE TO RELATED APPLICATIONS

This application is a bypass continuation of International Application No. PCT/US2021/060732, filed Nov. 24, 2021, which claims the benefit of U.S. Provisional Pat. Application No. 63/118,096, filed Nov. 25, 2020, the disclosures of each are incorporated by reference herein in their entireties.

### INCORPORATION OF SEQUENCE LISTING

The sequence listing that is contained in the file named “HAN0002-201BC1-US,” which is 23.1 kilobytes as measured in Microsoft Windows operating system and was created on May 19, 2023, is filed electronically herewith and incorporated herein by reference.

### FIELD OF THE DISCLOSURE

The present disclosure relates to virology. More particularly, the present disclosure related to methods and compositions for treating and preventing SARS-CoV-2 viral infection.

### BRIEF SUMMARY OF THE DISCLOSURE

In one aspect, the disclosure provides a recombinant polypeptide comprising: (a) a lactoferrin protein or fragment thereof; and (b) a SARS-CoV or SARS-CoV-2 Spike (S) protein or fragment thereof; and (c) a linker; wherein the lactoferrin protein or fragment thereof binds to HSPG on the host cell surface and the SARS-CoV or SARS-CoV-2 Spike (S) protein or fragment thereof binds to the ACE2 receptor on the host cell surface, wherein binding of both the lactoferrin or fragment thereof and the SARS-CoV or SARS-CoV-2 Spike (S) protein or fragment thereof prevents binding of a virus. In one embodiment, the lactoferrin comprises a wild-type lactoferrin protein or fragment thereof or a recombinant lactoferrin or fragment thereof. In other embodiments, the SARS-CoV-2 spike (S) protein sequence is set forth herein as SEQ ID NOs:1-2, and the lactoferrin comprises a sequence as set forth in SEQ ID NOs:3-4. In another embodiment, the virus is a virus from the Coronaviridae family. In another embodiment, the Coronaviridae virus is Severe Acute Respiratory Syndrome (SARS). In another embodiment, the severe acute respiratory syndrome (SARS) virus is SARS-CoV or SARS-CoV-2. In another embodiment, the disclosure provides a pharmaceutical composition comprising a recombinant polypeptide as described herein.

In another aspect, the disclosure provides a method of preventing or treating SARS-CoV-2 infection in a subject exposed to SARS-CoV-2, comprising administering to the subject a therapeutically or prophylactically effective amount of a pharmaceutical composition as described herein. In one embodiment, the pharmaceutical composition is administered via the oral, mucosal, nasopharyngeal, or parenteral routes. In another embodiment, the method further comprises a therapeutically effective amount of at least a second treatment.

In another aspect, the disclosure provides a method of preventing or treating SARS-CoV-2 infection in a subject exposed to SARS-CoV-2, comprising administering to the subject: (a) a prophylactically effective amount of a lactoferrin protein or fragment thereof; or (b) a prophylactically effective amount of a lactoferrin protein or fragment thereof and a prophylactically effective amount of ivermectin; or (c) a prophylactically effective amount of a recombinant lactoferrin protein or fragment thereof; or (d) a prophylactically effective amount of a recombinant lactoferrin protein or fragment thereof and a prophylactically effective amount of ivermectin.

In another aspect, the disclosure provides a method of treating an early stage SARS-CoV-2 infection comprising administering to the subject: (a) a therapeutically effective amount of a lactoferrin protein or fragment thereof; or (b) a therapeutically effective amount of a lactoferrin protein or fragment thereof and a therapeutically effective amount of ivermectin; or (c) a therapeutically effective amount of a recombinant lactoferrin protein or fragment thereof; or (d) a therapeutically effective amount of a recombinant lactoferrin protein or fragment thereof and a therapeutically effective amount of ivermectin.

In another aspect, the disclosure provides a method of treating an early stage SARS-CoV-2 infection comprising administering to the subject: (a) a therapeutically effective amount of a lactoferrin protein or fragment thereof; or (b) a therapeutically effective amount of a lactoferrin protein or fragment thereof and a therapeutically effective amount of ivermectin; or (c) a therapeutically effective amount of a recombinant lactoferrin protein or fragment thereof; or (d) a therapeutically effective amount of a recombinant lactoferrin protein or fragment thereof and a therapeutically effective amount of ivermectin. In one embodiment, the lactoferrin comprises a wild-type lactoferrin or a recombinant lactoferrin or fragment thereof. In one embodiment, the lactoferrin protein is human lactoferrin. In another embodiment, the lactoferrin protein binds to the cell membrane of cells in a subject and is taken into the cell upon binding. In another embodiment, the lactoferrin is present in the cytoplasm.

The present disclosure additionally sets forth a number of embodiments as described herein.

In some embodiments, the disclosure provides a recombinant polypeptide comprising: a) a lactoferrin protein or fragment thereof; b) a linker; and c) a SARS-CoV or SARS-CoV-2 Spike (S)-protein or fragment thereof; wherein the lactoferrin protein or fragment thereof binds to heparan sulfate proteoglycans (HSPG) on a host cell surface, wherein the S-protein or fragment thereof binds to the ACE2 receptor on the host cell surface, and wherein the recombinant polypeptide inhibits binding of a virus to the host cell.

In some embodiments, the Spike protein comprises a sequence as set forth in SEQ ID NO:1-2.

In some embodiments, the lactoferrin protein comprises a sequence as set forth in SEQ ID NOs:3-4.

In some embodiments, the linker comprises a sequence as set forth in SEQ ID NOs:5-6.

In some embodiments, the recombinant polypeptide comprises a sequence set forth in SEQ ID NO:7-8.

In some embodiments, the methods further comprise an immunoglobulin (Ig) Fc-domain.

In some embodiments, the recombinant polypeptide comprises a sequence set forth in SEQ ID NO:9-10.

In some embodiments, the virus is a virus from the Coronaviridae family.

In some embodiments, the Coronaviridae virus is Severe Acute Respiratory Syndrome (SARS).

In some embodiments, the severe acute respiratory syndrome (SARS) virus is SARS-CoV or SARS-CoV-2.

In some embodiments, the disclosure provides a pharmaceutical composition comprising: a) a lactoferrin protein or fragment thereof; b) a linker; and c) a SARS-CoV or SARS-CoV-2 Spike (S)-protein or fragment thereof; wherein the lactoferrin protein or fragment thereof binds to heparan sulfate proteoglycans (HSPG) on a host cell surface, wherein the S-protein or fragment thereof binds to the ACE2 receptor on the host cell surface, and wherein the recombinant polypeptide inhibits binding of a virus to the host cell.

In some embodiments, the disclosure provides a method of preventing or treating SARS-CoV-2 infection in a subject exposed to SARS-CoV-2, comprising administering to the subject a therapeutically or prophylactically effective amount of the pharmaceutical composition comprising: a) a lactoferrin protein or fragment thereof; b) a linker; and c) a SARS-CoV or SARS-CoV-2 Spike (S)-protein or fragment thereof; wherein the lactoferrin protein or fragment thereof binds to heparan sulfate proteoglycans (HSPG) on a host cell surface, wherein the S-protein or fragment thereof binds to the ACE2 receptor on the host cell surface, and wherein the recombinant polypeptide inhibits binding of a virus to the host cell.

In some embodiments, the pharmaceutical composition is administered via the oral, mucosal, nasopharyngeal, or parenteral routes.

In some embodiments, the methods further comprise a therapeutically effective amount of at least a second treatment.

In some embodiments, the second treatment comprises ivermectin, Remdesivir®, a monoclonal antibody, Regeneron®, hydroxychloroquine, Mornupiravir, and/or Paxlovid.

In some embodiments, the disclosure provides a method of preventing or treating SARS-CoV-2 infection in a subject exposed to SARS-CoV-2, comprising administering to the subject: (a) a prophylactically effective amount of a lactoferrin protein or fragment thereof; or (b) a prophylactically effective amount of ivermectin; or (c) a prophylactically effective amount of a lactoferrin protein or fragment thereof and a prophylactically effective amount of ivermectin; or (d) a prophylactically effective amount of a recombinant lactoferrin protein or fragment thereof; or (e) a prophylactically effective amount of a recombinant lactoferrin protein or fragment thereof and a prophylactically effective amount of ivermectin.

In some embodiments, the disclosure provides a method of treating or preventing an early stage SARS-CoV-2 infection comprising administering to the subject: (a) a therapeutically effective amount of a lactoferrin protein or fragment thereof; or (b) a therapeutically effective amount of a lactoferrin protein or fragment thereof and a therapeutically effective amount of ivermectin; or (c) a therapeutically effective amount of a recombinant lactoferrin protein or fragment thereof; or (d) a therapeutically effective amount of a recombinant lactoferrin protein or fragment thereof and a therapeutically effective amount of ivermectin.

In some embodiments, the disclosure provides a method of treating or preventing a late stage SARS-CoV-2 infection comprising administering to the subject: (a) a therapeutically effective amount of a lactoferrin protein or fragment thereof; or (b) a therapeutically effective amount of a lactoferrin protein or fragment thereof and a therapeutically effective amount of ivermectin; or (c) a therapeutically effective amount of a recombinant lactoferrin protein or fragment thereof; or (d) a therapeutically effective amount of a recombinant lactoferrin protein or fragment thereof and a therapeutically effective amount of ivermectin.

In some embodiments, the lactoferrin protein is human lactoferrin.

In some embodiments, the lactoferrin protein binds to the cell membrane of cells in a subject and is taken into the cell upon binding.

In some embodiments, the lactoferrin is present in the cytoplasm.

### BRIEF DESCRIPTION OF THE DRAWINGS

FIG. 1 - Shows that heparin binding proteins can be identified by amino acid sequences known as heparin binding motifs ‘XBBBXXBX’ and ‘XBBBXXBX’, where X is a hydropathic residue and B is a basic residue of arginine or lysine (Antiviral Research, 181, 10487:1-9, 2020). These heparin binding motifs are present in SARS-CoV-2 Spike protein and in lactoferrin as indicated in FIG. 1A and FIG. 1B, respectively. These heparin binding sites are conserved in lactoferrins from multiple species of mammals as indicated (box) in FIG. 1C. Lactoferrin from these species are believed to have anti SARS-CoV-2 activity similar to data shown in FIG. 3.

FIG. 2 - Shows the DNA (top, corresponding to SEQ ID NO:9) and protein (bottom, corresponding to SEQ ID NO:10) sequences of the lactoferrin (LF)-Fc-SARS-CoV-2-RBD recombinant polypeptide (LF-Fc-RBD). The IL2 leader is shown in capitalized underlined text; the linker is shown in lowercase, underlined, bold text; the human LF is shown in capitalized regular text; the Fc region is shown in capitalized, italicized text; the SARS-CoV-2 Spike RBD (Arg319-Phe541) is shown in lowercase, italicized text.

FIG. 3 - Shows the effects of lactoferrin on SARS-CoV-2 pseudovirus infection in VeroE6/TMPRSS2 cells. Lanes 2-4 (left to right) show human lactoferrin manufactured in rice. Lanes 5-7 show human lactoferrin from human milk.

FIG. 4 - Shows the DNA (top, corresponding to SEQ ID NO:7) and protein (bottom, corresponding to SEQ ID NO:8) sequences of the human lactoferrin (LF)-SARS-CoV-2-RBD recombinant polypeptide (hLF-CoV2-RBD). The signal peptide is shown in capitalized underlined text; the mature human LF is shown in capitalized regular text; the 4-Gly linker is shown in lowercase, underlined, bold text; the SARS-CoV-2 Spike RBD (Arg319-Phe541) is shown in capitalized italicized text.

FIG. 5 - Shows a human lactoferrin-Fc-SARS-CoV-2 RBD construct (hLF-Fc-CoV2RBD).

FIG. 6 - Demonstrates the combined effects of Lactoferrin (LF) and Ivermectin (IVM) on the infectivity of MLV-Spp in VeroE6/TMPRSS2 cells.

FIG. 7 - Shows infectivity of MLV-Spp in target cells (top) and VeroE6 cells stably transfected with TMPRSS2. Clone #7 was used as VeroE6/Tmprss2 target cells.

FIG. 8 - Shows infectivity of MLV-Spp in VeroE6 cells (top left), BHK/ACE2 cells (top right), 293/ACE2 cells (bottom left), and a comparison of all 3 cell types (bottom right).

FIG. 9 - Shows the DNA (top, corresponding to SEQ ID NO:3) and protein (bottom, corresponding to SEQ ID NO:4) sequences of human lactoferrin (LF). The signal peptide is underlined in both sequences.

FIG. 10 - Demonstrates hLF binding to the HSPG receptor on the cell membrane, which is inhibited by heparin. As shown in the right column, hLF staining on the cell membrane is absent when heparin is present.

FIG. 11 - Demonstrates that hLF enters VeroE6/Tmprss2 (VE6/T) cells when incubated with hLF at 2.5 µM, 10 µM, and 50 µM for 24 hrs and visualized using anti-LF antibodies.

FIG. 12 - Shows a western blot, demonstrating that hLF enters, accumulates, and remains intact in cells for up to the 24 hours tested. Cells were exposed to hLF at 2.5 µM, 10 µM, and 50 µM for 0.5, 2, 6, and 24 hours. LF was detected with anti-LF antibody (sc-53498) at 1:1,000 dilution for 20 minutes. LF found in cell lysate co-migrates with untreated control LF.

### BRIEF DESCRIPTION OF THE SEQUENCES

SEQ ID NO:1 - DNA sequence of SARS-CoV spike receptor binding domain (RBD) ligand.

SEQ ID NO:2 - Amino acid sequence of SARS-CoV spike receptor binding domain (RBD) ligand.

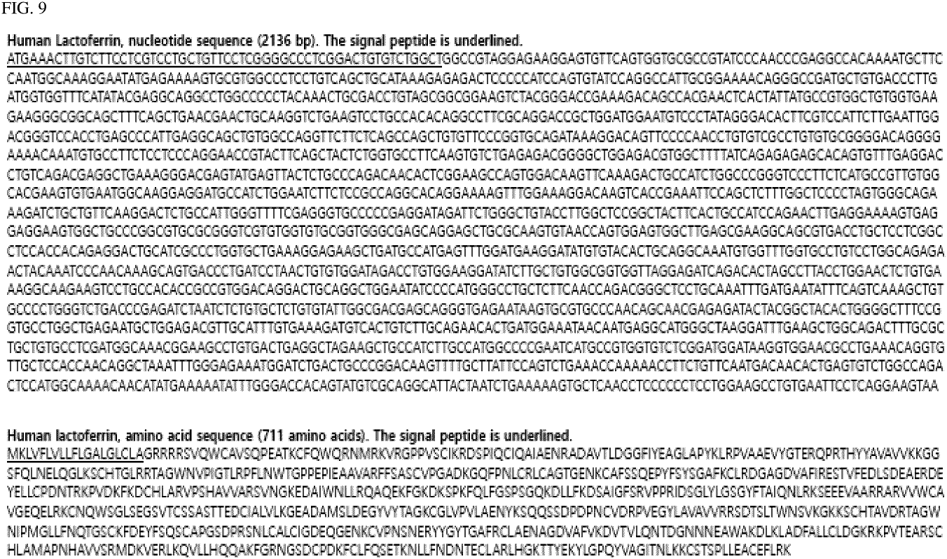

SEQ ID NO:3 - DNA sequence (2137 base pairs) of human lactoferrin (LF), corresponding to FIG. 9 (top). The sequence encoding the signal peptide in the FIG. is underlined.

SEQ ID NO:4 - Amino acid sequence (711 amino acids) of human lactoferrin (LF), corresponding to FIG. 9 (bottom). The signal peptide in the FIG. is underlined.

SEQ ID NO:5 - DNA sequence of 4-glycine (Gly) linker.

SEQ ID NO:6 - Amino acid sequence of 4-glycine (Gly) linker.

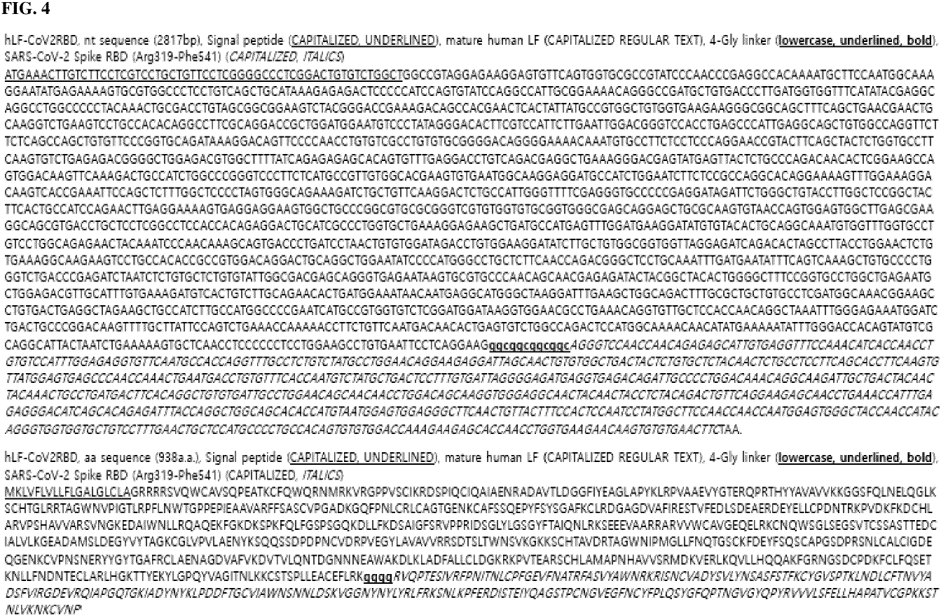

SEQ ID NO:7 - DNA sequence of hLF-CoV2-RBD construct, corresponding to FIG. 4 (top).

SEQ ID NO:8 - Amino acid sequence of hLF-CoV2-RBD construct, corresponding to FIG. 4 (bottom).

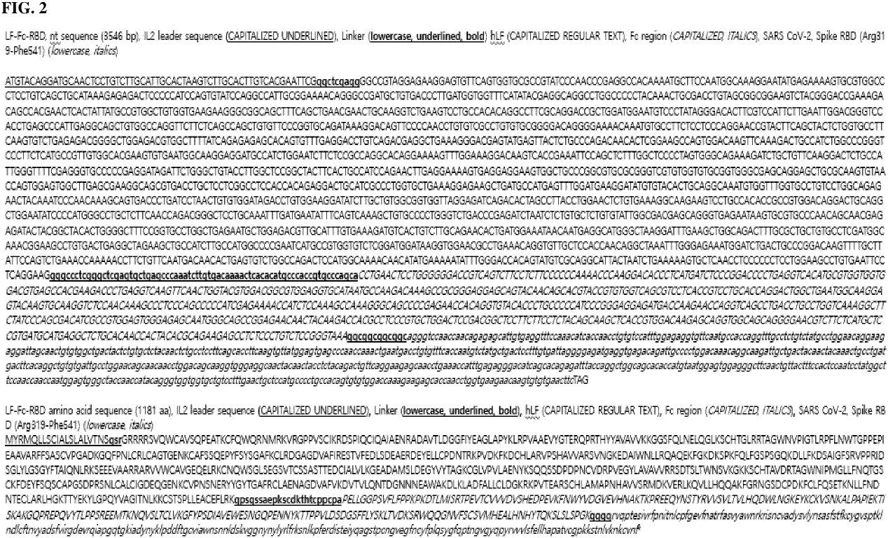

SEQ ID NO:9 - DNA sequence of LF-Fc-RBD construct, corresponding to FIG. 2 (top).

SEQ ID NO:10 - Amino acid sequence of LF-Fc-RBD construct, corresponding to FIG. 2 (bottom).

### DETAILED DESCRIPTION OF THE DISCLOSURE

Enveloped viruses enter cells by receptor-mediated endocytosis using their surface proteins, such as the spike (S) protein or the envelope (E or Env) proteins, which interact with a receptor on the surface of a host cell. The interaction between the Env protein and the cell surface receptor works in a “lock-and-key” fashion. Env proteins have one or more structural epitopes that are recognized by a binding pocket within the receptor(s). The 3D structure of an epitope is determined by the amino acid sequence. Certain viruses use only one cellular receptor, while others use more than one. For viruses that utilize more than one receptor, co-operative ligand interaction plays a key role and the binding process is likely sequential, i.e., binding of the first ligand induces binding of the second ligand. Examples of viruses in which this process occurs include HIV, in which binding of CCR5 opens up the binding site for CD4 binding, and HCV, in which binding of HSPG and/or SRB1 allows CD81 binding.

A second mechanism of viral binding to a viral receptor on the host cell is through charge interaction. The viral Env protein is known to have a positively charged domain, which is evolutionarily conserved. The negatively charged domains of cellular receptors are involved in this process. These sites can be a sole receptor for a virus to infect a cell, or can be a second receptor, i.e., a co-receptor. The charge of the binding pocket can be donated by acidic amino acids or by sulfation of the protein by, for example, housekeeping enzymes of the host. There are 2 known ways of sulfating protein, one of which occurs through tyrosine sulfation (as is the case for HIV), and the other which occurs by sugars such as heparan sulfate (HS), which is the case for HCV. It is not currently known exactly how many enveloped viruses use sugar-based sulfate groups for their receptor-mediated entry process.

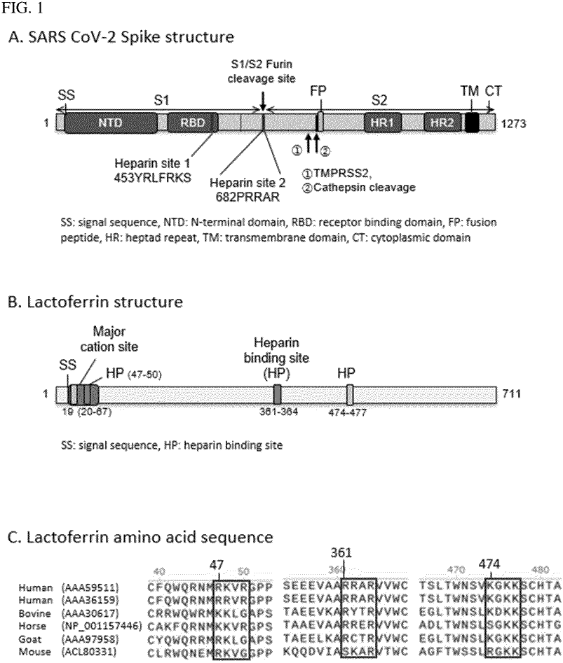

SARS-CoV-2 is the cause of the current COVID-19 global pandemic that has resulted in worldwide illness and death. SARS-CoV-2 is a positive-sense single-stranded RNA virus that is a member of the Coronaviridae family of viruses. It infects human cells by interactions between spike (S) glycoprotein and charged amino acid residues in the angiotensin converting enzyme 2 (ACE2) receptor. S proteins of many coronaviruses, including SARS-CoV-2, are cleaved into, and function as, two separate subunits, S1 (binds the receptor) and S2 (induces fusion between viral and cellular membranes) (see FIG. 1). This cleavage occurs by furin protease at the S1/S2 furin cleavage site during virion assembly and secretion in target cells. In contrast to this, the S protein of SARS-CoV is not cleaved, but rather functions as a single protein, even though the two proteins share approximately 70% homology between the two. The S protein of SARS-CoV-2 has a PRRAR pentapeptide insertion at the S1/S2 cleavage site, which makes the furin cleavage possible and is absent in the SARS-CoV S protein. The high arginine (R) content of the SARS-CoV-2 S protein leads to an expected higher charge interaction of the virus with HSPGR. The S1/S2 furin cleavage site is incomplete in producer cells. It is further cleaved by TMPRSS2, a host cell-derived serine protease, in the target cell during viral entry. The S2 protein is primed at an S2′ site by cathepsin in the endosome, which is activated low pH, and the fusion peptide is ejected as a result.

Many viruses enter their host cells by attaching themselves to one or more cell surface receptors. SARS-CoV-2 utilizes the ACE2 receptor for entry into cells, combined with heparan sulfate proteoglycans (HSPG) for attachment to the cell. HSPG is a housekeeping protein that is expressed in all cell types, while the ACE2 receptor is highly expressed in the endothelial lining of multiple organs, including the airways, lungs, gastrointestinal tract, heart, kidney, and liver, as well as the blood vessels. For this reason, COVID-19 is considered to be an endothelial disease cause by viral infection with SARS-CoV-2. The glycosaminoglycan component of HSPG is heparan sulfate (HS). Upon binding, the SARS-CoV-2 spike protein receptor-binding domain undergoes a structural change. SARS-CoV-2 may use CD147 as an attachment receptor, which is found in the lungs, but is expressed much higher in red blood cells (RBCs) and the vascular endothelium. CD147 is also found in T cells, however its function in T cells is unknown but potentially linked to immune suppression. Based on the above, the present inventors have identified that administration of lactoferrin to a patient infected with SARS-CoV-2 mitigates entry of the virus into the host cell. SARS-CoV-2 infection down-regulates the ACE2 receptor in the endothelium lining in multiple organs, including lung, heart, blood vessels, liver, and kidney, causing endothelial inflammation and thrombosis, which is responsible for multiple organ failure in COVID-19 patients.

Thus, in some embodiments, the present disclosure provides recombinant polypeptides, pharmaceutical compositions, and related methods for preventing and treating SARS-CoV-2 infection comprising administration of a therapeutically effective amount of LF, either alone, or in combination with, ivermectin (IVM) to a patient infected with SARS-CoV-2. In some embodiments, a lactoferrin for use as described herein may be present in a recombinant polypeptide as described herein, wherein a lactoferrin or fragment thereof is fused to the receptor binding domain of the SARS-CoV-2 spike (S) protein. In some embodiments, such administration of LF, either wild-type LF or present in a recombinant fusion protein, results in amelioration of symptoms associated with COVID-19 in a patient. The present disclosure thus intends to encompass treatment for, and vaccination against any virus capable of infecting a cell using cell surface receptors.

Unless otherwise specified herein, the recombinant proteins, compositions, and/or methods described herein can be performed in accordance with the procedures exemplified herein or routinely practiced methods well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13: 978-0121821906); U.S. Pat. Nos. 4,965,343, and 5,849,954; Sambrook et al., Molecular Cloning: A Laboratory Manual, Cold Spring Harbor Press, N.Y., (3rd ed., 2000); Brent et al., Current Protocols in Molecular Biology, John Wiley & Sons, Inc. (2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the methods of the present disclosure.

Embodiments of the present disclosure provide a method of preventing or treating SARS-CoV-2 viral infection in a subject in need thereof, comprising administering to the subject a therapeutically or prophylactically effective amount of LF. LF is shown to improve pathology and mortality rate when used in COVID-19 infection. In some embodiments, LF may be used as a stand-alone therapy to treat SARS-CoV-2 infection in a patient. In other embodiments, LF may be used in combination with other available therapies, such as ivermectin (IVM), for treating when used in early infection. In other embodiments, LF, either alone, or in combination with other therapies, such as IVM, may be used as a prophylaxis, or preventative for SARS-CoV-2 infection.

Embodiments of the present disclosure provide a recombinant polypeptide comprising (a) a lactoferrin protein or fragment thereof; and (b) a SARS-CoV or SARS-CoV-2 Spike (S) protein or fragment thereof; and (c) a linker; wherein the lactoferrin protein or fragment thereof binds to HSPG on the host cell surface and the SARS-CoV or SARS-CoV-2 Spike (S) protein or fragment thereof binds to the ACE2 receptor on the host cell surface, wherein binding of both the lactoferrin or fragment thereof and the SARS-CoV or SARS-CoV-2 Spike (S) protein or fragment thereof prevents binding of a virus. Other embodiments provide methods of treating or preventing SARS-CoV-2 infection, or treating SARS-CoV-2 infection at both early and late stages, comprising administering to the subject: (a) a prophylactically effective amount of a lactoferrin protein or fragment thereof; or (b) a prophylactically effective amount of a lactoferrin protein or fragment thereof and a prophylactically effective amount of ivermectin; or (c) a prophylactically effective amount of a recombinant lactoferrin protein or fragment thereof; or (d) a prophylactically effective amount of a recombinant lactoferrin protein or fragment thereof and a prophylactically effective amount of ivermectin.

### Human Lactoferrin

Human lactoferrin is a 70-80 kDa basic protein with a pI of 8.7. Lactoferrin is a member of the transferrin family and is found in milk, blood, and exocrine secretions (tears, saliva, nose drops). The concentration of lactoferrin in milk and blood is about 10 µM. The lactoferrin protein has 2 globular domains: N-and C-globules, each having 2 domains, N1 and N2, or C1 and C2. Each globule has 1 glycosylation site and 1 metal binding site, which binds 2 ions of iron, zinc, and copper.

Lactoferrin has an iron affinity that is 30 times higher than transferrin under acidic pH conditions during inflammation (when lactic acid accumulates). It is found as an oligomer depending on the concentration of the protein (Ca is involved in this process). Lactoferrin is pyrimidine-dependent RNAse, and is a source of RNAse activity in milk, tears, and nasal droplets. It is also a double-stranded DNA (dsDNA) binding protein, used for dsDNA immobilization. Lactoferrin also has antiviral activity against a number of viruses, including, but not limited to, HSV, CMV, HIV, MLV, HCV, hantavirus, rotavirus, polio, RSV, and SARS-CoV. In addition, lactoferrin levels are up-regulated in patients infected with SARS-CoV infection (BMC Immunology 6:2, 2005). Once SARS-CoV-2 enters red blood cells, a viral protein ejects iron from heme groups in the cell, causing organ damage and leading to hypoximia and a rise in lactic acid levels. Lactoferrin stabilizes heme and scavenges the ejected toxic iron ions. For treatment of malaria, hydroxychloroquine inhibits polymerization of damaged heme caused by malaria infection. Damaged hemes release iron ions and lyse the infected cells. The Plasmodium prevents cell death by polymerizing hemes.

Lactoferrin may be taken up by cells upon binding to the HSPG present on the cell surface of a host cell, e.g., a human host cell. Once lactoferrin is taken into the cell, it can be detected in the cytoplasm. This interaction is inhibited by heparin such that no LF staining is seen on the cell membrane when heparin is present.

Lactoferrin is known to block SARS-CoV infection by binding to the HSPG receptor (PLoS ONE 6:e23710, 2011). The anchoring sites provided by HSPG on the cell surface permit initial contact between SARS-CoV and host cells. SARS-CoV rolls onto the cell membrane by binding to HSPG and scans for specific entry receptors, which leads to subsequent cell entry. Lactoferrin blocks the infection of SARS-CoV by binding to HSPG. Lactoferrin binds to cell-surface HSPG molecules and prevents the preliminary interaction between the virus and host cells, thereby preventing the subsequent internalization process. In vitro experiments using SARS-CoV pseudovirus to infect VeroE6 cells demonstrated that the addition of lactoferrin, or enzymatic removal of cell surface HSPG, prevents SARS pseudovirus entry.

In similar experiments, a recombinant lactoferrin-S protein fusion having a 70-amino acid ACE2 receptor binding site peptide demonstrated that S (and the 70-aa peptide) binds the ACE2 receptor away from its enzyme active site with no enzyme or cellular function changes. The results were proven using a small molecule inhibitor. This type of fusion protein enables both lactoferrin to bind to the HSPG on the cell surface and the receptor binding site on the S protein to bind to the ACE2 receptor on the cell surface, thereby tying up both the receptor and co-receptor on the host cell and preventing entry of the virus into the cell. In fact, the receptor binding domain of the SARS-CoV-2 S protein was shown to be as protective when used as a vaccine as the full-length S protein in macaques (Science 10:1126, May 2020). Thus, in some embodiments, a recombinant protein as described herein, wherein the receptor binding domain of the SARS-CoV-2 spike (S) protein is fused with lactoferrin, binds to the host cell surface receptors (e.g., ACE2 receptor and HSPG) and prevent binding of the SARS-CoV-2 virus from binding and entering the host cell. This approach is different from other studies in the art, in which recombinant proteins bind to the viral particles themselves to prevent attachment of the virus to the cell. The present disclosure rather discloses a recombinant protein that binds to the host cell and effectively ties up the cell surface receptors such that the virus particles can no longer bind and infect the cell.

Multiple homologs of lactoferrin are known and available in the art, with a number of conserved regions among these. For example, bovine lactoferrin shares 77% homology with human lactoferrin. An exemplary lactoferrin is set forth herein as SEQ ID NO:4, although as described herein, any lactoferrin protein or functional fragment thereof may be used in accordance with the present disclosure. Thus, use of lactoferrin or variants thereof for treatment of coronaviruses as described herein may encompass any lactoferrin, such as including, but not limited to, human lactoferrin, bovine lactoferrin, ovine lactoferrin, equine lactoferrin, or lactoferrin from any mammal or species producing lactoferrin. In addition, in some embodiments, a recombinant lactoferrin expressed or manufactured in other species, e.g., plant species such as rice (e.g., Ventria Bioscience) may be used and are encompassed within the scope of the present disclosure. Recombinant lactoferrin is an effective anti-viral drug, and is effective in the low µM range. As described herein, lactoferrin may be used as a stand-alone viral treatment for SARS-CoV-2, or may be used in a combination therapy with one or more other treatments or therapies, such as ivermectin as described herein. In other embodiments, a recombinant polypeptide as described herein may be administered alone to a patient for treatment of SARS-CoV-2, or may be used in a combination therapy with one or more other treatments or therapies, such as ivermectin. In other embodiments, a recombinant polypeptide may be combined with more than one drug treatment as described herein. Any combination of a recombinant polypeptide and one or more drugs as described herein may be used herein to treat SARS-CoV-2 infection.

As described herein, LF has antiviral activity against a number of viruses, including, but not limited to, HSV, CMV, HIV, MLV, HCV, hanta, rota, polio, RSV, and SARS-CoV. In some embodiments, any coronavirus may be treated or prevented using the methods and composition of the present disclosure, including, but not limited to, SARS-CoV or SARS-CoV-2. The levels of LF are up-regulated during SARS-CoV infection, (BMC Immunology, 2005, 6:2).

### Ivermectin

Avermectin was discovered from bacterium Streptomyces avermitilis in Japan, from which ivermectin, a derivative of greater potency and lower toxicity was introduced in 1981. Ivermectin has radically lowered the incidence of river blindness and lymphatic filariasis, as well as showing efficacy against an expanding number of other parasitic diseases. Ivermectin works by causing the invertebrate cell membrane to increase in permeability to chloride ions, resulting in cellular hyper-polarization, followed by paralysis and death.

As of 2019, ivermectin is available as a generic prescription drug in the US in a 3 mg tablet formulation. It is sold under several brand names in the US, including but not limited to, Heartgard®, Sklice, and Stromectol.

Ivermectin is also used in the prevention of malaria, as it is toxic to both the malaria plasmodium itself, and the mosquitos that carry it. Use of ivermectin at higher doses necessary to control malaria is probably safe, although large clinical trials have not yet been done.

Ivermectin has antiviral effects against several RNA viruses including ZKV, YFV, WNV. DENV, VEEV, CHIKV, SFV, SINV, Avian Influenza A virus (J Antibiotics 2020, 73:593-602). Ivermectin inhibits replication of SARS-CoV-2 in monkey kidney cell culture with an IC50 of 2.2 - 2.8 µM, making it a possible candidate repurposed drug for COVID-19 (Antiviral Res 2020, 178, 104787). Initially these doses used in cell culture were considered impractical for clinical use; however, the antiviral effect of ivermectin was soon demonstrated in patients with COVID-19 at lower clinical doses.

As of July, ivermectin was being studied in 19 ongoing and 18 planned clinical trials. Recently a clinical trial has been done with high risk healthcare workers to establish the efficacy and safety of ivermectin for prophylaxis of COVID-19. Two doses of ivermectin prophylaxis at dose of 300 ug/kg with a gap of 3 days was associated with 73% reduction of COVID-19 infection among healthcare workers (medRxiv 1101/2020).

The likely antiviral mechanism of action for ivermectin includes the suppression of a host cellular process, specifically the inhibition of nuclear transport by importin α/β1(Antiviral Res, 2020, 177, 104760), or inhibition of SARS-CoV-2 3-CL viral protease (Nature, 2021, 4:93, 1-10). Ivermectin is also implicated for the inhibition of SARS-CoV-2 virus binding to its potential cellular receptor CD147 (bioRxiv), although no evidence for a direct SARS-CoV spike binding to it was also reported (bioRxiv doi.org/10.1101/2020.07.25. 221036). Ivermectin is an FDA-approved drug used to treat a variety of parasites. Ivermectin targets SARS-CoV-2 spike (S) protein at CD147 binding sites, inhibiting the entry of the virus into RBC and T-cells. Ivermectin also acts as a viral replication inhibitor by inhibiting the host importin alpha/beta-1 nuclear transport proteins, which are part of a key intracellular transport process that viruses hijack to enhance infection by suppressing the host antiviral response.

As described herein, the Inventors have identified new prophylactic and therapeutic treatments for SARS-CoV-2 infection using combinations of LF and ivermectin (IVM). For example, a LF protein or fragment thereof may be administered to a patient exposed to or experiencing symptoms of SARS-CoV-2 infection (i.e., COVID-19 disease). In other embodiments, a recombinant fusion protein may be produced with the SARS-CoV-2 receptor binding domain (RBD) and used either alone or in combination with LF. From these experiments, it was determined that LF is an efficient inhibitor for SARS-CoV-2 infection in target cells (VeroE6 or VeroE6/Tmprss2), and inhibition is efficient for SARS-CoV-2 as it has increased affinity for HSPG (doi.org/10.1016/j.antiviral.2020.104873).

### Recombinant Polypeptides for Prevention of Viral Infection

In some embodiments, the present disclosure provides a recombinant polypeptide comprising: recombinant polypeptide comprising: (a) a lactoferrin protein or fragment thereof; and (b) a SARS-CoV or SARS-CoV-2 Spike (S) protein or fragment thereof; and (c) a linker; wherein the lactoferrin protein or fragment thereof binds to HSPG on the host cell surface and the SARS-CoV or SARS-CoV-2 Spike (S) protein or fragment thereof binds to the ACE2 receptor on the host cell surface, wherein binding of both the lactoferrin or fragment thereof and the SARS-CoV or SARS-CoV-2 Spike (S) protein or fragment thereof prevents binding of a virus.

Such a recombinant polypeptide binds to proteins present on the host cell surface, mimicking binding of a virus to its cell surface receptor on the host cell. In some embodiments, a recombinant polypeptide may have more than one polypeptide fragment, allowing the recombinant protein to co-operatively bind to more than one receptor on the host cell surface (e.g., ACE2 receptor and/or HSPG). In this way, the cell surface receptors are bound and unavailable for binding by a viral particle, effectively preventing entry of a virus into a cell and reducing or preventing the infectivity of the virus.

In some embodiments, a fusion molecule as described herein may comprise one or more elements, such as a lactoferrin protein or binding portion thereof, and the receptor binding domain (RBD) of the SARS-CoV-2 spike (S) protein. In some embodiments, a fusion protein as described herein may comprise a lactoferrin protein or variant or portion thereof, and a SARS-CoV-2 spike protein or portion thereof. In some embodiments, the LF and the S protein or S protein RBD may be separated by a linker, such as a 4-Glycine (Gly) linker. An exemplary linker is provided herein as SEQ ID NOs:5-6. In some embodiments, synthetic and/or genetically engineered variants of these elements are encompassed within the scope of the present disclosure.

To increase the activity or half-life of any recombinant polypeptide or compositions comprising these, a LF protein or fragment thereof and/or an S protein RBD as described herein may be fused or bound to a larger molecule or carrier. For example, the LF or fragment thereof and the S protein RBD may be fused to all or part of an immunoglobulin (Ig) Fc-domain, such as a construct comprising a human lactoferrin protein fused to an Ig Fc domain and a SARS-CoV-2 RBD as set forth in SEQ ID NOs:9-10 and shown in FIG. 2. Such a fusion confers on the recombinant polypeptide antibody effector functions, including the ability to mediate antibody-dependent cell-mediated cytotoxicity, to access mucosal compartments, and to transport across the placenta.

Thus, in some embodiments, a recombinant polypeptide as described herein may contain an Fc binding region of an immunoglobulin, in addition to the cell surface receptors present in the recombinant fusion polypeptide. In some embodiments, a recombinant polypeptide of the present disclosure may also contain portions of immunoglobulin molecules or antibodies, such as including, but not limited to, all or portions of a constant heavy chain, a variable heavy chain, a constant light chain, a variable light chain, a hinge region, and/or an Fc domain of a Ig, as well as variants thereof. For example, a recombinant polypeptide as described herein may be combined with portions of a human IgG. Any type of immunoglobulin may be used as appropriate, such as including IgG, IgA, IgM, IgD, IgE, and variants thereof.

For example, a recombinant polypeptide as described herein may be constructed by joining a LF or fragment thereof and/or a viral S protein RBD or fragment thereof at either the N-terminus or the C-terminus of an Ig-Fc fragment. The elements of the recombinant polypeptide may be joined directly together in any configuration, or they may be separated by a spacer or linker region. Such a spacer or linker region may be beneficial in some embodiments for proper placement of the viral S protein or fragments and the LF such that they are able to bind sequentially or co-operatively to the host cell surface receptors, and/or to ensure proper function of each of the components. Elements of a recombinant polypeptide as described herein may be joined in any order. In some embodiments, a LF protein or fragment thereof may be joined to a SARS-CoV-2 spike (S) receptor binding domain as described herein to produce a recombinant LF-SARS-CoV-2 spike RBD fusion protein. In some embodiments, the SARS-CoV-2 spike RBD protein may be joined to the C-terminal end of the LF protein. As would be understood by one of skill in the art, components herein that have been identified as useful in accordance with the disclosure for inclusion in a recombinant polypeptide or fusion protein may be altered in a number of ways to include variants having any cell surface receptor and/or co-receptor for any virus as disclosed herein, and any useful regions of any of the protein elements or fragments thereof. Such recombinant polypeptides and variants thereof are also encompassed within the scope of the disclosure.

### Combination Therapies for Treatment of Viral Infection

In some embodiments, the disclosure provides methods for treating a viral infection as described herein. In some embodiments, such a method may be useful for preventing or treating SARS-CoV-2 infection in a subject exposed to SARS-CoV-2, comprising administering to the subject: (a) a prophylactically effective amount of a lactoferrin protein or fragment thereof; or (b) a prophylactically effective amount of a lactoferrin protein or fragment thereof and a prophylactically effective amount of ivermectin; or (c) a prophylactically effective amount of a recombinant lactoferrin protein or fragment thereof; or (d) a prophylactically effective amount of a recombinant lactoferrin protein or fragment thereof and a prophylactically effective amount of ivermectin. In some embodiments, such a method may treat early stage infection of SARS-CoV-2, or may treat late stage infection of SARS-CoV-2 as described herein.

In some embodiments, a lactoferrin protein useful as described herein is a human lactoferrin protein. In some embodiments, the lactoferrin protein binds to the cell membrane of cells in a subject and is taken into the cell upon binding. As shown and described herein, the lactoferrin that is taken up by the cell is present and detectable in the cytoplasm.

Such methods involve administration of a recombinant polypeptide or a therapeutic composition as described herein to a patient or subject in need. In other embodiments, a method of the present disclosure for treating or preventing SARS-CoV or SARS-CoV-2 is provided. Such a method involves administering to the subject a therapeutically or prophylactically effective amount of a pharmaceutical composition as described herein and at least a second treatment. As would be understood by one of skill in the art, any drug for treating viral infection may be used as a second treatment or therapy. Examples of such drugs are described herein and may include, but are not limited to, e.g., ivermectin, Remdesivir®, Regeneron®, hydroxychloroquine, Mornupiravir, Paxlovid or any other anti-viral drugs deemed appropriate. Based on the SARS-CoV or SARS-CoV-2 virus cell entry mechanism, drugs that may be useful for treating SARS-CoV or SARS-CoV-2 infection may include, but are not limited to, inhibitors of TMPRSS2 serine protease and inhibitors of angiotensin-converting enzyme 2 (ACE2).

Blocking binding of ACE2, the host cell receptor for the S protein of SARS-CoV-2, and/or inhibition of TMPRSS2 (e.g., with camostat mesylate (Foipan™), a clinically proven and commercial serine protease inhibitor that partially blocks infection by SARS-CoV and HCoV-NL63 in HeLa cell expressing ACE2 and TMPRSS2, or with nafamostat mesylate (Buipel™), which is a synthetic serine protease inhibitor), which is required for viral S protein priming may prevent cell entry of SARS-CoV-2. Other drugs known in the art for treatment of SARS-CoV or SARS-CoV-2 infection may include chloroquine phosphate (Resochin™) and hydroxychloroquine (Quensyl™, Plaquenil™, Hydroquin™, Dolquine™, and Quinoric™), as well as off-label antiviral drugs, such as the nucleotide analogue remdesivir, HIV protease inhibitors lopinavir and ritonavir, broad-spectrum antiviral drugs arbidol and favipiravir, as well as antiviral phytochemicals known and available in the art may limit the spread of SARS-CoV-2 and the morbidity and mortality of the current COVID-19 pandemic. ACE inhibitors, standard drugs for the treatment of hypertension and chronic heart failure, have thus far not proven to be as useful for treating SARS-CoV or SARS-CoV-2, as they do not appear to inhibit ACE2 receptor, although a number of other drugs and compounds have been shown to inhibit ACE2 receptor. Any of these drugs are encompassed within the scope of the present disclosure. In some embodiments, a drug as described herein, including, but not limited to, the drugs listed above, as well as lactoferrin, azithromycin, zinc, sialic acid, among others, may be used to treat a viral infection as described herein. In some embodiments, any drug known in the art to treat virus infection, specifically SARS-CoV or SARS-CoV-2, may be used, such as including, but not limited to, chloroquine, hydroxychloroquine, remdesivir, ritonavir/lopinavir (Kaletra™, camostat mesilate, nafamostat mesilate (Buipel™), cepharanthine/selamectin/mefloquine hydrochloride, lopinavir/ritonavir (Kaletra™), Favipiravir (Avigan™), Umifenovir (Arbidol™), 3Clpro, flavonoids such as luteolin, myricetin, apigenin, quercetin, kaempferol, baicalin, wogonoside, Emodin, Regeneron, Resveratrol, Mornupiravir, and/or Paxlovid.

In some embodiments, a drug as described herein for treatment of SARS-CoV or SARS-CoV-2, e.g., lactoferrin and/or ivermectin, may be combined with a recombinant polypeptide as described herein. In other embodiments, a drug as described herein may be combined with another drug as described herein for combination therapy. For example, in non-limiting embodiments, lactoferrin may be combined with one or more of ivermectin, azithromycin, zinc, and remdesivir. In other embodiments, azithromycin may be combined with zinc and remdesivir. In accordance with the present disclosure, any drug treatment may be combined with any other drug treatment as described herein, or may be combined with a recombinant polypeptide as described herein. A number of drug treatments described herein are known and available in the art. The present disclosure provides a novel treatment for SARS-CoV and SARS-CoV-2 comprising administration of a recombinant polypeptide as described herein and at least a second drug treatment or therapy such as one or more of the drugs described herein.

In some embodiments, a recombinant polypeptide or composition thereof as described herein may be combined with one or more combinations of, e.g., lactoferrin, ivermectin, azithromycin, zinc, remdesivir, or the like. For example, in some embodiments, a recombinant polypeptide or composition thereof as described herein may be combined with lactoferrin and/or ivermectin. In some embodiments, LF may be co-administered to a patient with a recombinant polypeptide as described herein, or with another drug such as ivermectin. As described herein, LF and/or IVM may be administered to a patient in a single dose, or in multiple doses. As dosages of LF and IVM are available in the art, any appropriate dose of LF and/or IVM may be administered to a subject as deemed appropriate by a clinician.

In some embodiments, a dose of LF may include, but is not limited to, a single dose of about 200 µg/kg. In some embodiments, a second or further dose is given to the subject as described herein. In some embodiments, a dose of LF may be about 100 mg (0.1 g) to about 5 g per day, including about 0.1 g, about 0.2 g, about 0.3 g, about 0.4 g, about 0.5 g, about 0.6 g, about 0.7 g, about 0.8 g, about 0.9 g, about 1.0 g, about 1.1 g, about 1.2 g, about 1.3 g, about 1.4 g, about 1.5 g, about 1.6 g, about 1.7 g, about 1.8 g, about 1.9 g, about 2.0 g, about 2.1 g, about 2.2 g, about 2.3 g, about 2.4 g, about 2.5 g, about 2.6 g, about 2.7 g, about 2.8 g, about 2.9 g, about 3.0 g, about 3.1 g, about 3.2 g, about 3.3 g, about 3.4 g, about 3.5 g, about 3.6 g, about 3.7 g, about 3.8 g, about 3.9 g, about 4.0 g, about 4.1 g, about 4.2 g, about 4.3 g, about 4.4 g, about 4.5 g, about 4.6 g, about 4.7 g, about 4.8 g, about 4.9 g, about 5.0 g, or the like.

In some embodiments, a dose of LF may include, but is not limited to, a single dose of about 200 µg/kg. In some embodiments, a second or further dose is given to the subject as described herein. In some embodiments, a dose of IVM may be 600 µg/kg or 1200 µg/kg per day for a specific number of consecutive days, e.g., 2, 3, 4, 5, 6, 7, 8, 9, 10, or the like. Thus, in some embodiments, a dose of IVM appropriate for treatment of SARS-CoV-2 infection may be about 100 µg/kg, or about 200 µg/kg, or about 300 µg/kg, or about 400 µg/kg, or about 500 µg/kg, or about 600 µg/kg, or about 700 µg/kg, or about 800 µg/kg, or about 900 µg/kg, or about 1000 µg/kg, or about 1100 µg/kg, or about 1200 µg/kg, or about 1300 µg/kg, or about 1400 µg/kg, or about 1500 µg/kg, or about 1600 µg/kg, or about 1700 µg/kg, or about 1800 µg/kg, or about 1900 µg/kg, or about 2000 µg/kg, or the like.

### Expression Systems and Vectors Encoding a Recombinant Polypeptide

As detailed herein, the disclosure provides pharmaceutical and therapeutic compositions that can be administered to a mammalian subject in need of long-term in vivo protection against or treatment for viral infection. Such compositions typically contain expression systems, e.g., polynucleotide sequences, expression vectors, or viral vectors that encode or express a recombinant polynucleotide as described herein. Compositions of the present disclosure allow optimal in vivo activity or co-expression in a subject or patient (e.g., human or non-human primate) of a recombinant polypeptide as described herein, which provides potent and long-term protection against infection of a virus as described herein.

Optimal expression of a recombinant polypeptide as described herein can be accomplished via various mechanisms. Such optimal expression may be accomplished using a desired structural design of an expression vector encoding a recombinant polypeptide, or by the use of appropriate regulatory elements in an expression vector. In addition, optimal expression of a recombinant polypeptide of the disclosure in vivo may further be optimized by measurement of cellular levels of the recombinant polypeptide as described herein. Any assays for determination of appropriate levels of the polypeptide may be used as appropriate. Such tests can all be readily carried out via standard assays or protocols well known in the art. In other embodiments, viral neutralizing activities may be assessed using any assays known in the art, such as a neutralization assay.

In some preferred embodiments, polynucleotide sequences encoding a recombinant polypeptide as described herein are operably-linked to expression control sequences (e.g., promoter sequences) in a virus-based expression vector or expression system described herein. Some examples of viral vectors suitable for the recombinant proteins and methods described herein include, but are not limited to, retrovirus-based vectors, e.g. lentiviruses, adenoviruses, adeno-associated viruses (AAV), vaccinia vectors, α-virus vectors, measles virus vectors (MSV), or vesicular stomatitis vectors (VSV). In some embodiments, an adenoviral vector that may be useful for the present disclosure may be Ad5, Ad26. In some embodiments, a composition of the disclosure can contain a recombinant AAV vector (rAAV) or viral particle harboring a vector expressing a recombinant polypeptide as described herein. In some embodiments, a vaccinia vector useful for the present disclosure may be a Canary Pox vector. In some embodiments, the structure of the vector may be modified as necessary for optimization of expression or to achieve a desired cellular level, of the recombinant polypeptide, such as including expression controlling elements (e.g., promoter or enhancer sequences).

Various promoter sequences well known in the art may be used in accordance with the disclosure. These include, but are not limited to, e.g., CMV promoter, elongation factor-I short (EFS) promoter, chicken-actin (CBA) promoter, EF-la promoter, human desmin (DES) promoter, Mini TK promoter, and human thyroxine binding globulin (TBG) promoter. Additionally, an expression vector of the disclosure may include a number of regulatory elements to achieve optimal expression of the recombinant polypeptide. For example, a 5′-enhancer element and/or a 5′-WPRE element may be included to elevate expression of the recombinant polypeptide. WPRE is a post-transcriptional response element that has 100% homology with base pairs 1093 to 1684 of the Woodchuck hepatitis B virus (WHYS) genome. When used in the 3′ UTR of a mammalian expression cassette, it can significantly increase mRNA stability and protein yield. As used herein, an “expression cassette” refers to a polynucleotide sequence comprising at least a first polynucleotide sequence capable of initiating transcription of an operably linked second polynucleotide sequence and optionally a transcription termination sequence operably linked to the second polynucleotide sequence. As used herein, an expression cassette may comprise an exogenous nucleic acid encoding a recombinant polypeptide as described herein operably linked to a promoter as described herein.

By expressing a recombinant polypeptide as described herein in a subject or patient, effective and long term in vivo protection against and/or treatment of viral infection in subjects such as humans. For such a method, a subject may be administered a pharmaceutical composition that contains a therapeutically or pharmaceutically effective amount of a recombinant polypeptide or therapeutic composition or expression system of the disclosure. In some related embodiments, the disclosure provides therapeutic compositions that contain expression systems for optimally expressing a recombinant polypeptide as described herein in the subject. The expression systems may be polynucleotide sequences or expression vectors, as well as liposomes or other lipid-containing complexes, and other macromolecular complexes capable of mediating delivery of a polynucleotide sequence to a host cell or subject. Various expression vectors or systems can be employed for expressing a recombinant polypeptide of the disclosure upon administration to a subject. In some embodiments, the expression vectors or expression systems may be based on viral vectors. In some other embodiments, the expression systems are comprised of polynucleotide sequences harboring coding sequences for a recombinant polypeptide as described herein, including deoxyribonucleic acid and ribonucleic acid sequences. In some embodiments, the expression vectors or systems are administered to subjects in the form of a recombinant virus. For example, the recombinant virus can be a recombinant adeno-associated virus (AAV), e.g., a self-complementary adeno-associated virus (scAAV) vector. Such viral delivery methods allow safe, unobtrusive, and sustained expression of high levels of protein therapeutics.

As described above, when using the therapeutic compositions of the disclosure for preventing or treating viral infections in a subject, expression levels of the recombinant polypeptide may be examined during the treatment process. In some embodiments, the administered recombinant polypeptides or compositions result in expression of the recombinant polypeptide in the subject in an amount that is sufficient to reduce the number of copies of viral RNA detectable in the plasma of the subject by at least 2-, 3-, 4-, 5-, 6-, 7-, 8-, 9-, 10-, 15-, 20-, 25-, 30-, 35-, 40-, 45-, 50-, 55-, 60-, 65-, 70-, 75-, 80-, 85-, 90-, 95-, 100-, 150-, 200-, 250-, 300-, 350-, 400-, 450-, 500-fold, 750-fold, 1000-fold, or more. In some preferred embodiments, treatment of a subject or patient with a recombinant polypeptide or a therapeutic or pharmaceutical composition of the disclosure results in a reduction of viral RNA to undetectable levels in the blood or plasma of the treated subject. Such undetectable levels may be defined as fewer than 50 copies of viral RNA per mL of plasma in a real-time reverse transcriptase polymerase chain reaction (real-time RT PCR) assay.

An expression vector as described herein may contain the coding sequences and other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors may be modified to provide such functionalities. Selectable markers can be positive, negative, or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Expression vectors or systems suitable for the disclosure include, but are not limited to, isolated polynucleotide sequences, e.g., plasmid-based vectors which may be extra-chromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/ DOPE, DOGS/DOPE or DMRIE/DOPE liposomes, and/or associated with other molecules such as DNA-anti-DNA antibody-cationic lipid (DOTMA/DOPE) complexes. Exemplary gene viral vectors are known in the art and described below. Vectors may be administered via any route including, but not limited to, intramuscular, buccal, rectal, intravenous or intracoronary administration, and transfer to cells may be enhanced using electroporation and/or iontophoresis.

Some embodiments can employ adeno-associated virus vectors or adenoviral vectors for optimally expressing a recombinant polypeptide as described herein in a subject or patient. Adenoviral vectors may be made replication-incompetent by deleting the early (El A and El B) genes responsible for viral gene expression from the genome. They may be stably maintained into the host cells in an extrachromosomal form. These vectors have the ability to transfect both replicating and nonreplicating cells. Adeno-associated virus vectors refer to recombinant adeno-associated viruses (rAAV) that are derived from nonpathogenic parvoviruses. They evoke essentially no cellular immune response and produce transgene expression lasting months in most systems. Like adenovirus, adeno-associated virus vectors also have the capability to infect replicating and nonreplicating cells and are believed to be nonpathogenic to humans. Pharmaceutical or Therapeutic Compositions for Preventing Viral Infection

In some embodiments, the disclosure provides a therapeutic or pharmaceutical composition comprising a live viral expression vector and a polynucleotide sequence expressing a recombinant polypeptide as described herein. Viral vectors are described in detail above and would be known to one of skill in the art. In some embodiments, an expression vector as described herein may be an adenoviral vector, a vaccinia vector, an α-virus vector, a measles virus vector (MSV), or a vesicular stomatitis vector (VSV). Other vectors known and available in the art may also be used as described herein.

In some embodiments, a recombinant polypeptide as described herein may be provided as a pharmaceutical or therapeutic composition to be administered to a subject or patient. A composition of the present disclosure may comprise a recombinant polypeptide as described herein in a single unit, or alternatively, in some embodiments a recombinant polypeptide as described herein may comprise two or more components or subunits that separately bind to the viral proteins to prevent entry into a cell. In some embodiments, the different components, e.g., peptides or polynucleotide chains, may be conjugated covalently or noncovalently prior to administration to a subject or patient. In some embodiments, a recombinant polypeptide as described herein may contain multiple distinct polypeptide chains (e.g., immunoglobulin heavy chains and a light chains). In some embodiments, one or more of the polypeptide chains may be required in order to bind to a host cell surface receptor.

In some embodiments, a recombinant polypeptide as described herein may be provided or administered to a subject or patient as a fully assembled fusion protein. Alternatively, in some embodiments a recombinant polypeptide as described herein may comprise two or more components or subunits that separately bind to the viral proteins to prevent entry into a cell. In some embodiments, the different components, e.g., peptides or polynucleotide chains, may be conjugated covalently or noncovalently prior to administration to a subject or patient. For embodiments of the disclosure wherein a live viral vector is provided, such a vector may encode a recombinant fusion protein as a single entity, or may encode separate, distinct components or subunits that are able to assemble in vivo into a recombinant polypeptide as described herein.

The disclosure provides pharmaceutical compositions and related methods of using the therapeutic compositions or expression systems for inhibiting, preventing, or treating viral infections. Also provided is a use of the polynucleotides, polypeptides, and expression vectors or systems described herein for the manufacture of a medicament to prevent or treat viral infections. The pharmaceutical composition can be either a therapeutic formulation or a prophylactic formulation. Typically, a pharmaceutical composition may contain one or more active ingredients and, optionally, some inactive ingredients. In some embodiments, the active ingredient may be a recombinant polypeptide, an expression vector, or an expression system as described herein. In some other embodiments, the active ingredient may include other antiviral agents in addition to the expression system of the disclosure. The composition may additionally include one or more pharmaceutically acceptable vehicles and, optionally, other therapeutic ingredients (for example, antibiotics or antiviral drugs). Various pharmaceutically acceptable additives may also be used in such compositions.

In some embodiments, an expression system in a pharmaceutical composition as described herein may contain an expression vector or a type of viral particle that may optimally express a recombinant polypeptide as described herein. In general, the amount of vector(s) or viral particles administered to achieve a particular outcome will vary depending on various factors including, but not limited to, the gene and promoter chosen, the condition, patient-specific parameters, e.g., height, weight, and age, and whether prevention or treatment is to be achieved. A vectors or viral particle of the disclosure may conveniently be provided in the form of formulations suitable for administration, e.g., into the blood stream (e.g., in an intracoronary artery). A suitable administration format may best be determined by a medical practitioner or clinician for each patient individually, according to standard procedures.

A pharmaceutical composition of the disclosure may be prepared in accordance with standard procedures well known in the art. See, e.g., Remingtons Pharmaceutical Sciences, 19th Ed., Mack Publishing Company, Easton, Pa., 1995; Sustained and Controlled Release Drug Delivery Systems, J. R. Robinson, ed., Marcel Dekker, Inc., New York, 1978; U.S. Pat. Nos. 4,652,441; 4,917,893; 4,677,191; 4,728,721; and 4,675,189. Pharmaceutical compositions of the disclosure may be readily employed in a variety of therapeutic or prophylactic applications for preventing or treating viral infections. For subjects at risk of developing a viral infection, a vaccine composition of the disclosure may be administered to provide prophylactic protection against viral infection. Depending on the specific subject and conditions, a composition of the disclosure may be administered to a subject or patient by a variety of administration modes known to the person of ordinary skill in the art, for example, intramuscular, subcutaneous, intravenous, intra-arterial, intra-articular, intraperitoneal, oral, mucosal, nasopharyngeal, or parenteral routes. Nasal sprays provide an effective way to administer a prophylactic or therapeutic treatment as described herein. In some embodiments, a composition as described herein may be administered to a subject in need of such treatment for a time and under conditions sufficient to prevent, inhibit, and/or ameliorate a selected disease or condition or one or more symptom(s) thereof. For therapeutic applications, a composition may contain a therapeutically effective amount of the expression system described herein. For prophylactic applications, a composition as described herein may contain a prophylactically effective amount of an expression system as described herein. The appropriate amount of the expression system (expression vectors or viral particles) may be determined based on the specific disease or condition to be treated or prevented, severity, age of the subject, and other personal attributes of the specific subject (e.g., the general state of the subject’s health and the robustness of the subject’s immune system). Determination of effective dosages may additionally be guided with animal model studies (i.e., primate, canine, or the like), followed by human clinical trials, and by administration protocols that significantly reduce the occurrence or severity of targeted disease symptoms or conditions in the subject.

For prophylactic applications, a composition as described herein may be provided in advance of any symptom, for example in advance of infection. A prophylactic administration of the immunogenic compositions may serve to prevent or ameliorate any subsequent infection. Thus, in some embodiments, a subject to be treated is one who has, or is at risk for developing, a viral infection, for example because of exposure or the possibility of exposure to the virus. Following administration of a therapeutically effective amount of the disclosed therapeutic compositions, a subject or patient may be monitored for viral infection, symptoms associated with viral infection, or both.

For therapeutic applications, a composition as described herein may be provided at or after the onset of a symptom of disease or infection, for example after development of a symptom of viral infection, or after diagnosis of infection. A composition as described herein may thus be provided prior to the anticipated exposure to virus so as to attenuate the anticipated severity, duration or extent of an infection and/or associated disease symptoms, after exposure or suspected exposure to the virus, or after the actual initiation of an infection.

In some embodiments, a vector or viral particle of the disclosure may be provided in a dosage form containing an amount of a vector effective in one or multiple doses. For viral vectors, an effective dose may be any range deemed appropriate by a clinician or practitioner. Administration of a recombinant polypeptide, vector, viral particle, expression system, or composition may be in a buffer, such as phosphate buffered saline, or other appropriate buffer or diluent. The amount of buffer or diluent may vary and would be determined by a clinician or practitioner. For delivery of plasmid DNA alone, or plasmid DNA in a complex with other macromolecules, the amount of DNA to be administered would be an amount that results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered. For delivery of a recombinant polypeptide of the disclosure, an amount administered would be an amount that results in a beneficial effect to the recipient. For example, from 0.0001 to 100 g or more, e.g., up to 1 g, in individual or divided doses, e.g., from 0.001 to 0.5 g, or 0.01 to 0.1 g, of recombinant polypeptide can be administered.

In some embodiments, a composition of the disclosure may be combined with other agents known in the art for treating or preventing viral infections. These may include any drug known or available in the art for treating a viral infection, e.g., antibodies or other antiviral agents such as nucleoside reverse transcriptase inhibitors, non-nucleoside reverse transcriptase inhibitors, protease inhibitors, and fusion protein inhibitors. Administration of a composition and one or more known anti-viral agent may be either concurrently or sequentially.

Dosages of lactoferrin and ivermectin may be any dosage deemed appropriate by a physician. For example,

### RNA Molecules for Preventing Viral Infection

In some embodiments, the disclosure provides a RNA molecule for treatment or prevention of a viral infection. Such a RNA molecule may comprise a first ribonucleotide sequence expressing an Internal Ribosome Entry Site (IRES); and second ribonucleotide sequence expressing a recombinant polypeptide as described herein.

As used herein, an “IRES” refers to a RNA element or a region of an RNA molecule that is able to recruit the eukaryotic ribosome to the mRNA. An IRES allows for initiation of protein translation without requiring a 5′ cap for assembly of the initiation complex. Thus, in some embodiments, introduction of an IRES to a RNA molecule as described herein enables production of a recombinant polypeptide of the disclosure in a cap-independent manner, as part of the greater process of protein synthesis. In eukaryotic translation, initiation of protein translation typically occurs at the 5′ end of an mRNA molecule. In some embodiments, an IRES included in a RNA molecule as described herein enables the recombinant polypeptide to be translated by the cells of the subject or patient to whom such a nucleic acid molecule or composition thereof is administered. A T7 promoter (such as provided in SEQ ID NO:19) may be added upstream of an IRES. Large-scale RNA production can be accomplished in vitro using, for example, a T7 polymerase. RNA may be injected subcutaneously in saline with or without liposome. The injected RNA will be translated within the cells of the patient and the resulting translated recombinant polypeptide is secreted.

A recombinant polypeptide as described herein may contain multiple distinct polypeptide chains (e.g., immunoglobulin heavy chains and a light chains). In some embodiments, one or more of the polypeptide chains may be required in order to bind to a host cell-surface receptor protein or fragment thereof.

### Methods for Preventing or Treating SARS-CoV-2 Infection

In some embodiments, the disclosure provides a method of preventing or treating SARS-CoV-2 infection in a subject in need thereof, comprising administering to the subject a therapeutically or prophylactically effective amount of a pharmaceutical composition comprising LF as described herein. Such a method may comprise administration of any dose of LF effective for ameliorating or treating symptoms of SARS-CoV-2 infection.

A method of the present disclosure may treat or prevent infection of a subject or patient with SARS-CoV-2 as described herein. Administration of a composition comprising LF, alone, or in combination with IVM, as described herein may be in a clinical setting as described herein, or may be in an alternate setting as deemed appropriate by a clinician or practitioner. Further embodiments for administration of such compounds or polypeptides are described herein elsewhere.

In some embodiments, such a composition comprising LF may be combined with other therapies or treatments, such as IVM, for treatment of SARS-CoV-2 infection in a patient. Administration of LF, or co-administration of LF with another drug treatment such as IVM may reduce the number of days of COVID-19 symptoms by one or more days, such as reducing symptoms by 1, 2, 3, 4, 5, 6, 7, 8, 9, 10, 11, 12, 13, 14, 15, or the like. In other embodiments, symptoms of COVID-19 may be reduced by one week or more, such as including, but not limited to, one week, 2 weeks, 3 weeks, 4 weeks, 5 weeks, 6 weeks, 7 weeks, or 8 weeks, or more. In other embodiments, administration of LF, or co-administration of LF with another drug treatment such as IVM may reduce the severity or duration of COVID-19 symptoms by 10%, or 20%, or 30%, or 40%, or 50%, or 60%, or 70%, or 80%, or 90%, or 100%.

### Expression of Nucleic Acids

Polynucleotides useful in the present disclosure can be provided in an expression construct. Expression constructs of the disclosure generally include regulatory elements that are functional in the intended host cell in which the expression construct is to be expressed. Thus, a person of ordinary skill in the art can select regulatory elements for use in, for example, bacterial host cells, yeast host cells, mammalian host cells, and human host cells. Regulatory elements used for expression of nuclear genes include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, the term “operably linked” refers to a juxtaposition of the components described wherein the components are in a relationship that permits them to function in their intended manner. In general, operably linked components are in contiguous relation.

An expression construct of the disclosure can comprise a promoter sequence operably linked to a polynucleotide sequence encoding a polypeptide of the disclosure. Promoters can be incorporated into a polynucleotide using standard techniques known in the art. Multiple copies of promoters or multiple promoters can be used in an expression construct of the disclosure. In a preferred embodiment, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

Nuclear Expression constructs of the disclosure may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides of the disclosure. Classical enhancers are cis-acting elements that increase gene transcription and can also be included in the expression construct. Classical enhancer elements are known in the art, and include, but are not limited to, the cytomegalovirus (CMV) early promoter enhancer element, and the SV40 enhancer element. Intron-mediated enhancer elements that enhance gene expression are also known in the art. These elements must be present within the transcribed region and are orientation dependent.

DNA sequences that direct polyadenylation of mRNA transcribed from the expression construct can also be included in the expression construct, such as an SV40 poly A signal, and include, but are not limited to, an octopine synthase or nopaline synthase signal.

Polynucleotides of the present disclosure can be composed of either RNA or DNA, or hybrids thereof. The present disclosure also encompasses those polynucleotides that are complementary in sequence to the polynucleotides disclosed herein. Polynucleotides and polypeptides of the disclosure can be provided in purified or isolated form.

### Nucleic Acids

Any number of methods well known to those skilled in the art can be used to isolate and manipulate a DNA molecule. For example, as previously described, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. The polynucleotide sequence includes both the sense and antisense strands either as individual strands or in the duplex.

### Kits

The disclosure further provides a kit comprising one or more single-use containers comprising a recombinant polypeptide as described herein. In some embodiments, a kit of the disclosure may provide a viral vector for administration to a subject or patient. In some embodiments, a kit may provide a pharmaceutical composition comprising a recombinant polypeptide as described herein, for administration to a subject or patient. In other embodiments, sterile reagents and/or supplies for administration of a recombinant polypeptide, RNA, viral vector, and/or pharmaceutical composition as described herein, may be provided as appropriate. A kit may further comprise reagents for cell transformation and/or transfection, viral and/or cell culture, or both.

Components provided in a kit of the disclosure may include, for example, any starting materials useful for performing a method as described herein. Such a kit may comprise one or more such reagents or components for use in a variety of assays, including for example, nucleic acid assays, e.g., PCR or RT-PCR assays, luciferase (Luc) assays, cell transformation/transfection, viral/cell culture, blood assays, i.e., complete blood count (CBC), viral titer/viral load assays, antibody assays, viral antigen detection assays, viral DNA or RNA detection assays, virus neutralization assays, genetic complementation assays, or any assay useful in accordance with the disclosure. For viral strains that result in genetic or genomic alterations or mutations in the hose, such as retroviruses, certain genotyping assays for identification of viral sequences within a host genome may be useful and are encompassed within the disclosure. Components may be provided in lyophilized, desiccated, or dried form as appropriate, or may be provided in an aqueous solution or other liquid media appropriate for use in accordance with the disclosure.

Kits useful for the present disclosure may also include additional reagents, e.g., buffers, substrates, antibodies, ligands, detection reagents, media components, such as salts including MgCl₂, a polymerase enzyme, deoxyribonucleotides, ribonucleotides, expression vectors, and the like, reagents for DNA isolation, DNA/RNA transfection, or the like, as described herein. Such reagents or components are well known in the art. Where appropriate, reagents included with such a kit may be provided either in the same container or media as a primer pair or multiple primer pairs. In some embodiments, such reagents may be placed in a second or additional distinct container into which an additional composition or reagents may be placed and suitably aliquoted. Alternatively, reagents may be provided in a single container means. A kit of the disclosure may also include packaging components, instructions for use, including storage requirements for individual components as appropriate. Such a kit as described herein may be formulated for use in a clinical setting, such as a hospital, treatment center, or clinical setting, or may be formulated for personal use as appropriate.

### Definitions

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 CFR § 1.822 is used.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

Conservative amino acid substitutions providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); Arginine (R), Lysine (K); 5) Isoleucine (I), Leucine (L), Methionine (M), Valine (V); and 6) Phenylalanine (F), Tyrosine (Y), Tryptophan (W). Not all residue positions within a protein will tolerate an otherwise “conservative” substitution. For instance, if an amino acid residue is essential for a function of the protein, even an otherwise conservative substitution may disrupt that activity, for example the specific binding of an antibody to a target epitope may be disrupted by a conservative mutation in the target epitope.

In some embodiments, conservative amino acid substitutions, e.g., substituting one acidic or basic amino acid for another, can often be made without affecting the biological activity of a recombinant polypeptide as described herein. Minor variations in sequence of this nature may be made in any of the peptides disclosed herein, provided that these changes do not substantially affect (e.g., by 15% or more) the ability of the peptide or fusion polypeptide to neutralize the entry of a virus into its host cells.

As used herein, a “co-receptor” refers to a receptor that binds or is bound subsequent to, or concurrently with, a primary or first receptor that interacts with a viral Env protein. As used herein, a “receptor” may be a sole receptor for entry of a virus into a cell, or may be a secondary receptor, i.e., a co-receptor. A receptor is present on a host cell and interacts or binds with a viral Env protein. Entry of viruses such as enveloped viruses into host cells requires an envelope glycoprotein binding cooperatively to a host cell surface receptor and a co-receptor.

As used herein, “epitope” refers to an antigenic determinant. Epitopes are particular chemical groups or peptide sequences on a molecule that are antigenic, such that they elicit a specific immune response, for example, an epitope is the region of an antigen to which B and/or T cells respond. Epitopes may be formed both from contiguous amino acids or noncontiguous amino acids juxtaposed by tertiary folding of a protein.

As used herein, an “effective amount” of a compound, drug, vaccine or other agent refers to an amount that is sufficient to generate a desired response, such as reduce or eliminate a sign or symptom of a condition or disease. For instance, as described herein, an effective amount may be an amount necessary to inhibit viral entry into a host cell, or to inhibit viral replication, or to measurably alter outward symptoms of a viral infection. In general, this amount will be sufficient to measurably inhibit virus replication or infectivity. When administered to a subject, a dosage will generally be used that will achieve target tissue concentrations (for example, in lymphocytes) that has been shown to achieve in vitro inhibition of viral entry or replication. In some examples, an “effective amount” is one that treats (including prophylaxis) one or more symptoms and/or underlying causes of any of a disorder or disease. In one example, an effective amount is a therapeutically effective amount. In one example, an effective amount is an amount that prevents one or more signs or symptoms of a particular disease or condition from developing.

As used herein, a “fusion protein” refers to a recombinant polypeptide or protein containing amino acid sequence from at least two unrelated proteins that have been joined together, via a peptide bond, to make a single protein. The unrelated amino acid sequences can be joined directly to each other or they can be joined using a linker sequence. As used herein, proteins are unrelated, if their amino acid sequences are not normally found joined together via a peptide bond in their natural environment(s) (e.g., inside a cell). For example, as described herein, the amino acid sequences of one or more host cell surface receptors, such as a recombinant lactoferrin protein fused to the spike protein RBD, are not normally found joined together via a peptide bond.

As used herein, “gene delivery” refers to the introduction of an exogenous polynucleotide into a cell for gene transfer, and may encompass targeting, binding, uptake, transport, localization, replicon integration and expression.

As used herein, “gene transfer” refers to the introduction of an exogenous polynucleotide into a cell which may encompass targeting, binding, uptake, transport, localization and replicon integration, but is distinct from and does not imply subsequent expression of the gene.

As used herein, “gene expression” or “expression” refers to the process of gene transcription, translation, and post-translational modification.

As used herein, “subject” or “patient” refers to any animal classified as a mammal, e.g., human and non-human mammals. Examples of non-human animals include dogs, cats, cattle, horses, sheep, pigs, goats, rabbits, etc. Unless otherwise noted, the terms “patient” or “subject” are used herein interchangeably. In some embodiments, a subject amenable for therapeutic applications of the disclosure may be a primate, e.g., human and non-human primates.

As used herein, administration of a polynucleotide or vector into a host cell or a subject refers to introduction into the cell or the subject via any routinely practiced methods. This includes “transduction,” “transfection,” “transformation,” or “transducing,” as well known in the art. These terms all refer to standard processes for the introduction of an exogenous polynucleotide, e.g., a transgene in rAAV vector, into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and western blot, measurement of DNA and RNA by assays, e.g., northern blots, Southern blots, reporter function (Luc) assays, and/or gel shift mobility assays. Methods used for the introduction of the exogenous polynucleotide include well-known techniques such as viral infection or transfection, lipofection, transformation, and electroporation, as well as other non-viral gene delivery techniques. The introduced polynucleotide may be stably or transiently maintained in the host cell.

Transcriptional regulatory sequences of use in the present disclosure generally include at least one transcriptional promoter and may also include one or more enhancers and/or terminators of transcription. Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

The term “treating” or “alleviating” includes the administration of compounds or agents to a subject to prevent or delay the onset of the symptoms, complications, or biochemical indicia of a disease (e.g., a viral infection), alleviating the symptoms or arresting or inhibiting further development of the disease, condition, or disorder. Subjects in need of treatment include those already suffering from the disease or disorder, as well as those being at risk of developing the disease or disorder. Treatment may be prophylactic (to prevent or delay the onset of the disease, or to prevent the manifestation of clinical or subclinical symptoms thereof) or therapeutic suppression, or alleviation of symptoms after the manifestation of the disease.

A “vector” is a nucleic acid with or without a carrier that can be introduced into a cell. Vectors capable of directing the expression of genes encoding for one or more polypeptides are referred to as “expression vectors.” Examples of vectors suitable for the present disclosure include, e.g., viral vectors, plasmid vectors, liposomes, and other gene delivery vehicles.

As used herein, “domain” refers to a polypeptide that includes an amino acid sequence of an entire polypeptide or a functional portion of a polypeptide. Certain functional subsequences are known, and if they are not known, can be determined by truncating a known sequence and determining whether the truncated sequence yields a functional polypeptide.

As used herein, “expression construct” refers to a nucleic acid construct that includes an encoded exogenous nucleic acid protein that can be transcribed and translated for functioning in the recipient to which it was administered. In some embodiments, such an expression construct may comprise DNA sequences, RNA sequences, or combinations thereof. In some embodiments, such a construct may be genetically engineered into a vector appropriate for administration in a subject or patient, such as a human patient. For example, as described herein, a construct of the present disclosure may comprise a nucleic acid sequence encoding a recombinant polypeptide comprising: (a) a lactoferrin (LF) or fragment thereof, and (b) a SARS-CoV-2 spike (S) protein receptor binding domain (RBD).

In some embodiments, an expression construct may be provided to a subject or patient as a viral vector. Viral vectors are well known in the art and may be any viral vector appropriate for the present disclosure. For example, in some embodiments, a construct as described herein may include, but is not limited to, an adenoviral vector, an α-virus vector, a measles virus vector (MSV), or a vesicular stomatitis vector (VSV). One of skill in the art would be able to identify an appropriate viral vector for administration to a subject or patient, such as a human subject.

As used herein, “exogenous sequence” refers to a nucleic acid sequence that originates outside the host cell. An exogenous sequence may be a DNA sequence, an RNA sequence, or a combination thereof. Any type of nucleic acid available in the art may be used in accordance with the disclosure, as would be understood by one of skill in the art. Such a nucleic acid sequence can be obtained from a different species, or the same species, as that of the cell into which it is being delivered. In some embodiments, an exogenous nucleic acid sequence in accordance with the disclosure may encode a recombinant polypeptide as described herein, suitable for administration to a subject or patient. Such a recombinant polypeptide may be administered to a subject or patient in order to treat or prevent SARS-CoV-2 infection.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a,” and “an,” and “the,” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and can also cover other unlisted steps. Similarly, any composition or device that “comprises,” “has,” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1 Lactoferrin Is an Efficient Inhibitor of SARS-CoV-2 Infection

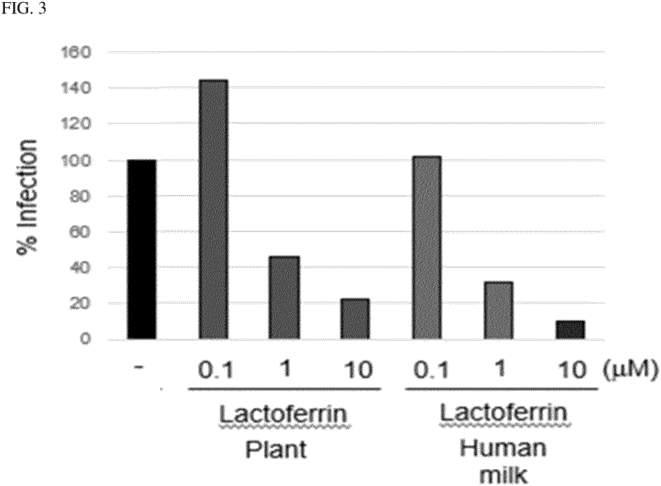

Lactoferrin was found to prevent entry of the SARS-CoV and SARS-CoV-2 viruses into cells in vitro. FIG. 3 shows the effects of plant lactoferrin (middle), and human lactoferrin isolated from human milk on infectivity of SARS-CoV-2 pseudovirus.

Administration of lactoferrin to a patient or subject having SARS-CoV or SARS-CoV-2 is not expected to produce any major side effects, and antiviral activity of lactoferrin would be unaffected by virus escape mutations. In addition, lactoferrin does not interfere with ACE2 receptor activity, as the protein binds away from the catalytic site. Lactoferrin would be expected to provide similar results for any virus of the Coronaviridae family, including, but not limited to, SARS-CoV and SARS-CoV-2.

Administration of lactoferrin is given to a patient or subject in one of a number of formulations, such as including, but not limited to oral, mucosal, nasopharyngeal, and/or parenteral. Scale up GMP production is available for such methods as described herein.

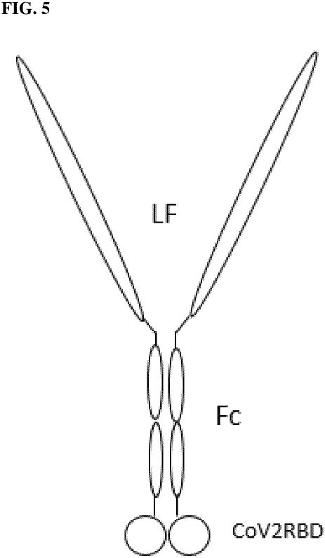

A lactoferrin-S protein fusion is capable of blocking both the HSPG and the ACE2 receptors on a host cell surface. A construct such as described herein (for example a construct shown in FIG. 1, FIG. 4, FIG. 5, or FIG. 2) or having a S protein (either full-length S protein, or the receptor binding domain alone) and a binding domain for the ACE2 receptor (i.e., co-operative binding) would increase affinity of receptor recognition.

### Example 2 Recombinant Polypeptides for Preventing SARS-CoV or SARS-CoV-2

In some embodiments, a recombinant protein may be produced that will bind to both viral receptors on a host cell surface, thereby preventing entry of the virus. For example, a recombinant protein is produced with (a) a lactoferrin protein or fragment thereof and (b) a SARS-CoV or SARS-CoV-2 Spike (S) protein or fragment thereof. In other embodiments, a recombinant protein is produced with (a) a lactoferrin protein or fragment thereof and (b) a SARS-CoV or SARS-CoV-2 Spike (S) protein receptor binding domain (RBD) or fragment thereof. In some embodiments, these 2 proteins or fragments thereof are joined together with a linker as described herein. FIGS. 1, 2, 4, and 5 provide exemplary constructs useful for treatment or prevention of SARS-CoV or SARS-CoV-2 as described herein.

For the Spike (S) protein, as described herein, the SARS-CoV-2 S protein is cleaved into 2 separate proteins, S1 and S2 at a PRRAR furin cleavage site (see FIG. 1). The S1 protein is involved in attachment of the virus to the host cell, and therefore, the full-length S protein may be used in such a recombinant polypeptide, or only the S1 portion or fragment of the S protein may be used, or only the specific amino acids that are involved in binding of the virus to its cell surface receptor (e.g., a 70-amino acid segment (Cell 181:1-10, Apr. 16, 2020).

Administration of such a recombinant polypeptide to a patient results in the lactoferrin portion of the recombinant polypeptide (or the lactoferrin fragment) binding to HSPG (viral anchoring sites) on the host cell surface. This binding leads to the co-operative binding of the S protein or fragment thereof binding to the ACE2 receptor on the cell surface. The binding of the lactoferrin and S protein components of the recombinant polypeptide prevent binding and fusion of the virus to the host cell.

A recombinant polypeptide as described above is administered to a patient having SARS-CoV-2 or symptoms thereof to prevent the entry of the virus into the cells and reduce the viral load in the patient, thereby treating the virus. Such a recombinant polypeptide is also administered to a patient having been exposed to SARS-CoV or SARS-CoV-2 for a prophylactic therapy.

A recombinant polypeptide or composition thereof as described herein may be combined with another drug treatment or therapy as described herein. For example, administration of a recombinant polypeptide of the present disclosure may be combined with a therapeutically effective amount of one or more of lactoferrin, azithromycin, zinc, and/or remdesivir, among others, for treatment of SARS-CoV or SARS-CoV-2 infection.

### Example 3 Prophylactic Treatment of SARS-CoV-2 After Exposure

In some embodiments, a patient who is exposed to SARS-CoV-2 is administered a prophylactically or therapeutically effective amount of lactoferrin (LF). Administration of LF to a patient exposed to SARS-CoV-2 prevents binding and subsequent infection of the cell by viral particles through the LF binding to cell-surface HSPG molecules and preventing the preliminary interaction between the virus and host cells. In this way, the LF prevents the subsequent internalization process of the virus thereby preventing infection. Such a preventative treatment is administered to a patient by a variety of routes, such as a nasal spray or inhaler. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is exposed to SARS-CoV-2 is administered a prophylactically or therapeutically effective amount of LF as described above in combination with ivermectin (IVM). Administration of LF and IVM to a patient exposed to SARS-CoV-2 prevents binding and subsequent infection of the cell by viral particles through the LF binding to cell-surface HSPG molecules and preventing the preliminary interaction between the virus and host cells. In this way, the LF prevents the subsequent internalization process of the virus thereby preventing infection. Such a preventative treatment is administered to a patient by a variety of routes, such as a nasal spray or inhaler. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is exposed to SARS-CoV-2 is administered a prophylactically or therapeutically effective amount of a recombinant polypeptide as described herein. A recombinant polypeptide useful in accordance with the present disclosure has (a) a LF protein or fragment thereof; and (b) a SARS-CoV or SARS-CoV-2 Spike (S) protein or fragment thereof; and (c) a linker, wherein the LF protein or fragment thereof binds to HSPG on the host cell surface and the SARS-CoV or SARS-CoV-2 Spike (S) protein or fragment thereof binds to the ACE2 receptor on the host cell surface, wherein binding of both the LF or fragment thereof and the SARS-CoV or SARS-CoV-2 Spike (S) protein or fragment thereof prevents binding of a virus. Administration of a recombinant LF (rLF) to a patient exposed to SARS-CoV-2 prevents binding and subsequent infection of the cell by viral particles through the rLF binding to cell-surface HSPG molecules and the rLF binding to the cell-surface ACE2 receptor molecules, preventing the binding of the virus to host cells and thereby preventing infection. Such a preventative treatment is administered to a patient by a variety of routes, such as a nasal spray or inhaler. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is exposed to SARS-CoV-2 is administered a prophylactically or therapeutically effective amount of a recombinant polypeptide as described above (i.e., rLF) in combination with IVM. Administration of a recombinant LF (rLF) to a patient exposed to SARS-CoV-2 prevents binding and subsequent infection of the cell by viral particles through the rLF binding to cell-surface HSPG molecules and the rLF binding to the cell-surface ACE2 receptor molecules, preventing the binding of the virus to host cells and thereby preventing infection. Such a preventative treatment is administered to a patient by a variety of routes, such as a nasal spray or inhaler. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

### Example 4 Treatment of Early Stage Infection of SARS-CoV-2

In some embodiments, a patient who is experiencing symptoms of early stage infection with SARS-CoV-2 is administered a therapeutically effective amount of LF. Administration of LF to a patient in early stage SARS-CoV-2 infection prevents binding and subsequent infection of the cell by additional viral particles through the LF binding to cell-surface HSPG molecules and preventing the interaction between the virus and host cells. In this way, the LF prevents the subsequent internalization process of additional virus, thereby preventing further infection. Such a treatment is administered to a patient by a variety of routes, such as an inhaler or intravenous (IV) injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is experiencing symptoms of early stage infection with SARS-CoV-2 is administered a therapeutically effective amount of LF as described above in combination with ivermectin (IVM). Administration of LF and IVM to a patient in early stage infection with SARS-CoV-2 prevents binding and subsequent infection of the cell by additional viral particles through the LF binding to cell-surface HSPG molecules and preventing subsequent interaction between the virus and host cells. In this way, the LF prevents the subsequent internalization process of the virus thereby preventing further infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is experiencing symptoms of early stage infection with SARS-CoV-2 is administered a therapeutically effective amount of a recombinant polypeptide as described herein. A recombinant polypeptide useful in accordance with the present disclosure has (a) a LF protein or fragment thereof; and (b) a SARS-CoV or SARS-CoV-2 Spike (S) protein or fragment thereof; and (c) a linker, wherein the LF protein or fragment thereof binds to HSPG on the host cell surface and the SARS-CoV or SARS-CoV-2 Spike (S) protein or fragment thereof binds to the ACE2 receptor on the host cell surface, wherein binding of both the LF or fragment thereof and the SARS-CoV or SARS-CoV-2 Spike (S) protein or fragment thereof prevents binding of a virus. Administration of a recombinant LF (rLF) to a patient with early stage SARS-CoV-2 infection prevents binding and subsequent infection of the cell by viral particles through the rLF binding to cell-surface HSPG molecules and the rLF binding to the cell-surface ACE2 receptor molecules, preventing the binding of the virus to host cells and thereby preventing further infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is experiencing symptoms of early stage infection with SARS-CoV-2 is administered a therapeutically effective amount of a recombinant polypeptide as described above (i.e., rLF) in combination with IVM. Administration of a recombinant LF (rLF) to a patient exposed to SARS-CoV-2 prevents binding and subsequent infection of the cell by viral particles through the rLF binding to cell-surface HSPG molecules and the rLF binding to the cell-surface ACE2 receptor molecules, preventing the binding of the virus to host cells and thereby preventing infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

### Example 5 Treatment of Late Stage Infection of SARS-CoV-2

In some embodiments, a patient who is experiencing symptoms of late stage infection with SARS-CoV-2 is administered a therapeutically effective amount of LF. Administration of LF to a patient in late stage SARS-CoV-2 infection prevents binding and subsequent infection of the cell by additional viral particles through the LF binding to cell-surface HSPG molecules and preventing the interaction between the virus and host cells. In this way, the LF prevents the subsequent internalization process of additional virus, thereby preventing further infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

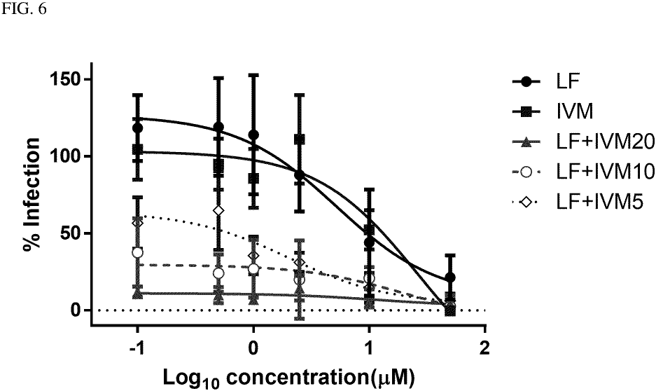

In some embodiments, a patient who is experiencing symptoms of late stage infection with SARS-CoV-2 is administered a therapeutically effective amount of LF as described above in combination with ivermectin (IVM). While FIG. 3 demonstrates the effects of plant and human lactoferrin isolated from human milk on infectivity of SARS-CoV-2 pseudovirus, FIG. 6 shows the combined effects of lactoferrin (LF) and ivermectin (IVM) on the infectivity of MLV-Spp in VeroE6/TMPRSS2 cells. Administration of LF and IVM to a patient in late stage infection with SARS-CoV-2 prevents binding and subsequent infection of the cell by additional viral particles through the LF binding to cell-surface HSPG molecules and preventing subsequent interaction between the virus and host cells. In this way, the LF prevents the subsequent internalization process of the virus thereby preventing further infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is experiencing symptoms of late stage infection with SARS-CoV-2 is administered a therapeutically effective amount of a recombinant polypeptide as described herein. A recombinant polypeptide useful in accordance with the present disclosure has (a) a LF protein or fragment thereof; and (b) a SARS-CoV or SARS-CoV-2 Spike (S) protein or fragment thereof; and (c) a linker, wherein the LF protein or fragment thereof binds to HSPG on the host cell surface and the SARS-CoV or SARS-CoV-2 Spike (S) protein or fragment thereof binds to the ACE2 receptor on the host cell surface, wherein binding of both the LF or fragment thereof and the SARS-CoV or SARS-CoV-2 Spike (S) protein or fragment thereof prevents binding of a virus. Exemplary such recombinant polypeptides are provided in FIG. 1, FIG. 4, FIG. 5, or FIG. 2. Administration of a recombinant LF (rLF) to a patient with late stage SARS-CoV-2 infection prevents binding and subsequent infection of the cell by viral particles through the rLF binding to cell-surface HSPG molecules and the rLF binding to the cell-surface ACE2 receptor molecules, preventing the binding of the virus to host cells and thereby preventing further infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary.

In some embodiments, a patient who is experiencing symptoms of late stage infection with SARS-CoV-2 is administered a therapeutically effective amount of a recombinant polypeptide as described above (i.e., rLF) in combination with IVM. Administration of a recombinant LF (rLF) to a patient with late stage SARS-CoV-2 infection prevents binding and subsequent infection of the cell by viral particles through the rLF binding to cell-surface HSPG molecules and the rLF binding to the cell-surface ACE2 receptor molecules, preventing the binding of the virus to host cells and thereby preventing infection. Such a treatment is administered to a patient by a variety of routes, such as an inhaler or IV injection. The patient is monitored for viral load, and reduction of symptoms of COVID-19 disease, as well as blood chemistry, such as including, but not limited to, complete blood count (CBC), respiratory volume, and chest x-ray or CT scan are performed as necessary. In some embodiments, LF reduces vascular endothelial inflammation by clearing Fe⁺² ions that are ejected from damaged RBC by SARS-CoV-2 infection.

### Example 6 Retrovirus-Based SARS-CoV-2 Pseudovirus Particles

Retrovirus (MLV)-based Sars-CoV-2 pseudovirus particles (pp), referred to herein as MLV-Spp, were prepared in order to test infection of host cells.

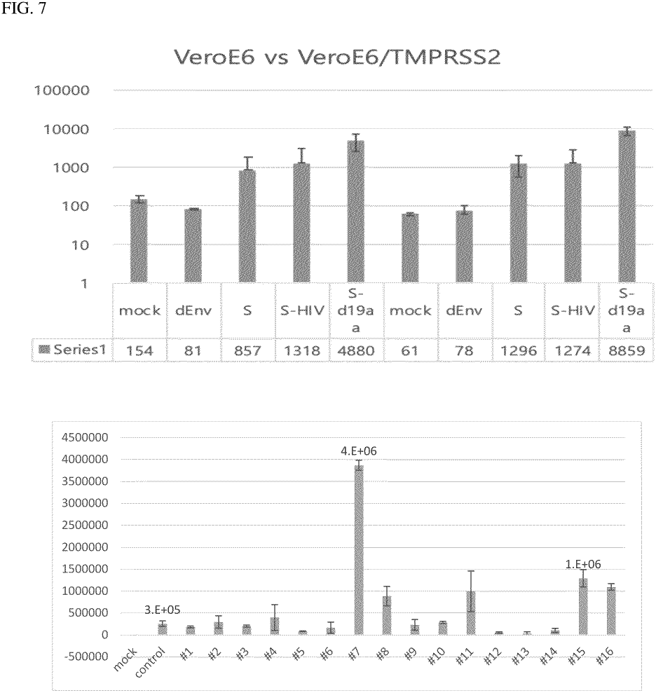

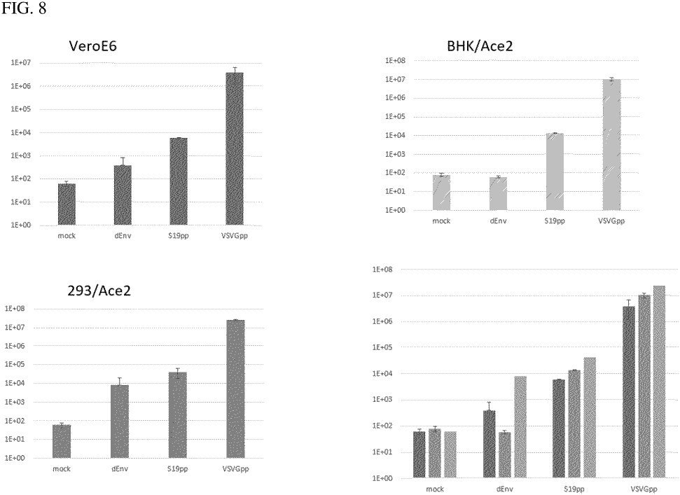

Pseudovirus infections were done in VeroE6 cells or in VeroE6/Tmprss2 co-transfected with plasmids encoding (1) the SARS S protein, (2) the gag/pol core protein, or (3) reporter. Packaging and budding of the viral particles resulted in pseudovirus particles expressing the SARS-CoV-2 S protein. FIG. 7 shows the results of an infectivity of MLV-Spp in target cells (top) and VeroE6 cells stably transfected with TMPRSS2. Clone #7 was used as VeroE6/Tmprss2 target cells. FIG. 8 shows results obtained for an infectivity assay of MLV-Spp in VeroE6 cells (top left), BHK/ACE2 cells (top right), 293/ACE2 cells (bottom left), and a comparison of all 3 cell types (bottom right).

Platinum-GP cells: 6×10⁶ cells/100 mm dish (5/18) using the following transfection conditions: 5 µg of GFP-Fluc, 2.5 µg (-, SD19 or VSV-G)-20 ul of LP3000.

The protocol was as follows:

Constructs and plasmids: The SARS-CoV-2 S glycoprotein sequence was taken from GenBank (Accession No. MN908947.3). The codon-optimized S protein cDNA sequence, cloned into pCMV plasmid, was purchased from Sino Biological (VG40589-UT) and is referred to herein as pS. The S protein cDNA with the C-terminal 19 amino acids deleted was synthesized by PCR and re-inserted into pCMV14 to create pS-d19. S protein cDNA with the cytoplasmic tail replaced with the HIV Env glycoprotein tail (CCSCGSCC) was synthesized and re-inserted into pCMV14 to create pS-hiv. A cDNA expression plasmid encoding serine protease Tmprss2 was purchased from Sino Biological and referred to pCMV-Tmprss2.

SARS-CoV-2 pseudotyped particles (Spp) were generated with a murine leukemia virus (MLV) core and luciferase reporter as previously described (J Visualized Expt 2019, 145:1-9). To this end, a packaging cell line Pt-gp was obtained, which expresses MLV gag and pol proteins (Cell Biolabs, #RTV 003), along with a transfer vector plasmid pBabe (Cell Biolab #RTV-001) that encodes a GFP reporter gene, an MLV Ψ-RNA packaging signal, and 5′-and 3′-flanking MLV long terminal repeat (LTR) regions. This vector was modified to include fruit fly luciferase (FLuc) and GFP reporter genes. A second plasmid was also used that encodes the SARS-CoV-2 S protein. These two plasmids were co-transfected into the packaging cells using Lipofectamine 3000 (Thermo) following the manufacturer’s protocol. Upon co-transfection, viral RNA and proteins get expressed within transfected cells allowing generation of pseudotyped particles (pp). Within these pp, the RNAs containing the luciferase gene reporter and packaging signal get encapsulated into nascent particles that bud out from cells into the culture medium with the S protein at their surface. The medium was harvested, cleared by centrifugation (700 x g for 15 min) for use in infectivity assays. Upon infection in target cells, the viral RNA containing the luciferase reporter and flanking LTRs gets released within the cell and the retroviral polymerase activities enable its reverse transcription into DNA and integration into the host cell genome. Quantification of the infectivity of pp in infected cells is then performed with a simple luciferase activity assay. Because the DNA sequence that gets integrated into the host cell genome only contains the luciferase gene and none of the MLV or coronavirus protein-encoding genes, they are inherently safer. SARS-CoV-2 Spp provide an excellent surrogate of native virions for studying viral entry into host cells.

Spp infection: 94-well plates were seeded with 1 × 10⁴ cells/well in 100 µl DMEM complete medium containing 10% FCS. The plate was incubated overnight (16-18 h) in a cell culture incubator with 5% CO₂ at 37° C. The cell culture supernatants were removed. Meanwhile, the pp were pre-incubated with test sample as indicated at 37° C. for 1 hr in 40 µl of complete medium. Cells were inoculated with 40 µl of pre-incubated pp solution. Cells were incubated in a 37° C., 5% CO₂ cell culture incubator for 2 h. 60 µl of pre-warmed (37° C.) DMEM-C medium was added to each well to adjust the volume to 100 µl. Cells were incubated in a 37° C., 5% CO₂ cell culture incubator for 48 h.

Infectivity Quantification: The Luciferase Assay System (Luciferase Assay system, Promega E4030) was used for evaluating infectivity. Luciferin substrate and 5x luciferase assay lysis buffer were thawed until they reached room temperature. Luciferase assay lysis buffer was diluted to 1× with sterile water. Supernatants of cells infected with pseudotyped particles were aspirated. 20 µL of 1× luciferase assay lysis buffer was added to each well. The plate was incubated on a rocker for 15 min at room temperature. Microcentrifuge tubes were prepared for each well by adding 20 µL of luciferin substrate in each tube. Luciferase activity measurement was performed one well at a time by transferring 2 µL of lysate to one tube containing 4 µL of luciferin substrate. The contents were mixed by gently flicking the tube to avoid displacing the liquid onto the walls of the tube. Luminescence was then measured using the luminometer and the relative light unit measurements were recorded.

Data Analysis: At the time of double transfection into packaging cells to produce pp, a mock transfection was performed, in which the second plasmid encoding the Spike coding sequence was deleted. This transfection did not produce pp, therefore the luciferase activity measurable from the target cells represented the background. This value was referred to as dEnv and was subtracted from the Luc value from samples for normalization. Normalized Luc values from transduced cells with pp alone were taken as 100% infection.

Calculation and plotting of relative luciferase unit averages and standard deviations: Using a graph plotting software, luciferase assay measurement averages and standard deviations of experimental and biological replicates were calculated. Data were plotted as a bar chart with standard deviations. For statistical analyses on data, at least three biological replicates were included in data sets.

Pseudotyped particle infection: Cells were observed under a light microscope for visual confirmation of a confluent carpet of cells. Cryovials of pseudotyped virus were thawed on ice. Cells were washed three times with 0.5 mL of pre-warmed (37° C.) DPBS.

Cell lines: Human embryonic kidney cell line 293 (#CRL-1573) and 293T expressing the SV40 T-antigen (#CRL-3216), human airway epithelial cell line Calu3 (#HTB-55), human alveolar epithelial cell line A549 (#CCL-185), human fibroblasts derived from lung tissue MRC5 (#CCL-171), African green monkey kidney cell line VeroE6 (#CRL-1586) and Vero 81 (#CCL-81) were obtained from ATCC (Manassas, VA, USA). All of the above cells were maintained in Dulbecco’s MEM containing 10% fetal bovine serum and 100 units penicillin, 100 µg streptomycin, and 0.25 µg Fungizone (1% PSF, Gibco) per milliliter. Rhesus monkey kidney cell line LLC-MK2 (#CCL-7) from ATCC was maintained in Opti-MEM containing 10% FBS and 1% PSF.

Production of SARS-CoV-2 S pseudovirions and virus entry: Pseudovirions were produced by co-transfection of 293T cells with psPAX2, pLenti-GFP, and plasmids encoding either SARS-CoV-2 S, SARS-CoV S, VSV-G, or empty vector by using polyetherimide (PEI). The supernatants were harvested at 40 and 64 h post-transfection, passed through a 0.45 µm filter, and centrifuged at 800 × g for 5 min to remove cell debris. To transduce cells with pseudovirions, cells were seeded into 24-well plates and inoculated with 500 µl media containing pseudovirions. After overnight incubation, cells were fed with fresh media. About 40 h post-inoculation, cells were lysed with 120 µl medium containing 50% Steady-glo (Promega) at room temperature for 5 min. The transduction efficiency was measured by quantification of the luciferase activity using a Modulus II microplate reader (Turner Biosystems, Sunnyvale, CA, USA). All experiments were done in triplicate and repeated at least twice or more.

Pseudovirus neutralization assay. SARS-CoV S, SARS-CoV-2 S, and VSV-G pseudovirions were pre-incubated with serially diluted either polyclonal rabbit anti-SARS S1 antibodies T62 or patient sera for 1 h on ice, then virus-antibody mixture was added onto 293/hACE2 cells in a 96-well plate. After 6 h incubation, the inoculum was replaced with fresh medium. Cells were lysed 40 h later and pseudovirus transduction was measured as previously described. Prior to experiments, patient sera were incubated at 56° C. for 30 min to inactivate complement.

### Example 7 Internalization of Lactoferrin Into the Cells

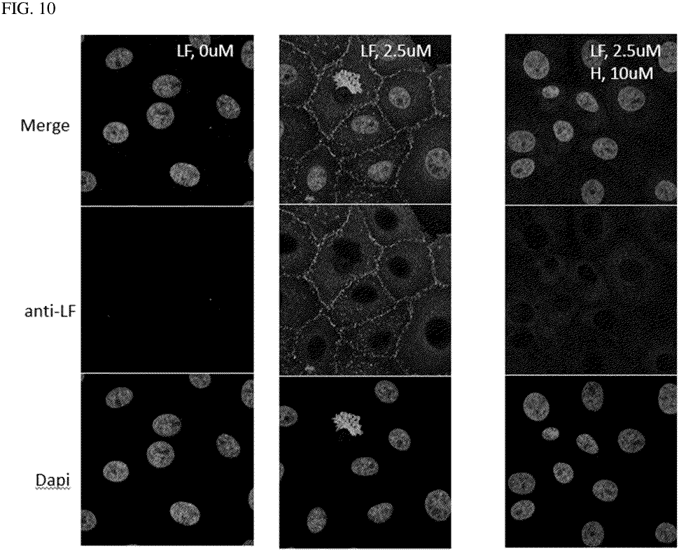

To visualize cell membrane localization of human lactoferrin (hLF), VeroE6/T cells (0.75×10⁵ cells/well) were chilled at 4° C. for 1 hr, incubated with 2.5 µM of hLF. Cells were washed, fixed using 4% PFA (Invitrogen), stained with anti-hLF antibody, and fluorescent image was taken under confocal microscope (FIG. 10, middle row). Cell nuclei were visualized using DAPI (FIG. 10, bottom row). To visualize the cell surface and nuclei at the same time, the confocal images above (i.e., FIG. 10, middle and bottom rows) were merged and are presented in FIG. 10, top row. Therefore, as evident in FIG. 10, hLF binds the HSPG receptor on the cell membrane, which is inhibited by heparin. To see the effect of heparin on the binding of hLF to the cell surface (specifically the HSPG receptor), cells were co-incubated with hLF and heparin (H), and the confocal image was taken (FIG. 10, right column).

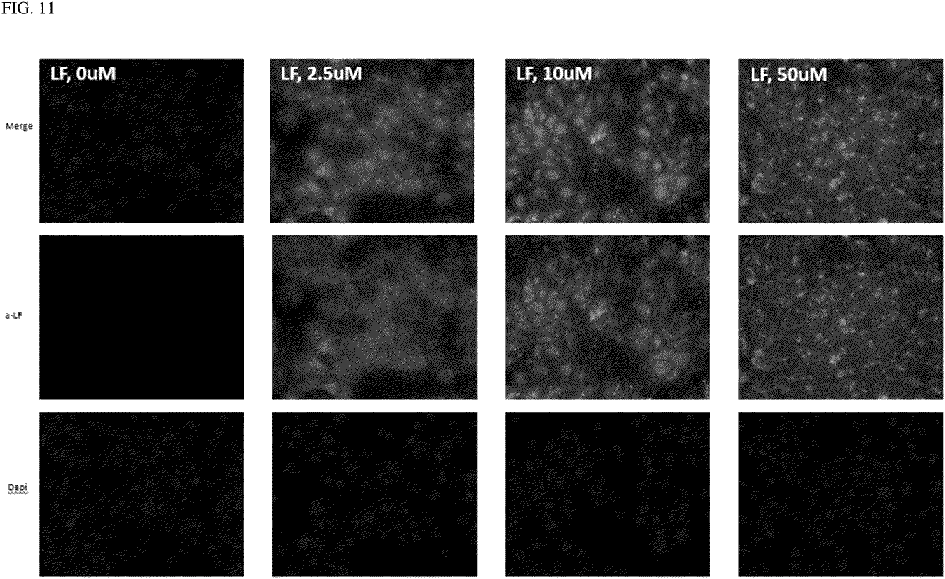

VE6/T cells in 24 well plate (5×10⁴ cells/well) were incubated with hLF at concentrations of 2.5 µM, 10 µM, and 50 µM for 24 hrs. Cells were washed, fixed with ice-cold methanol, blocked with 3% BSA in 0.5% triton X-100, PBS, stained with anti-LF antibody (sc-53498) as the primary antibody and Alexa 488 anti-mouse IgG (A21202, Invitrogen,) as the secondary antibody and images were taken using an immunofluorescent microscope. FIG. 11 demonstrates that hLF enters the cells.

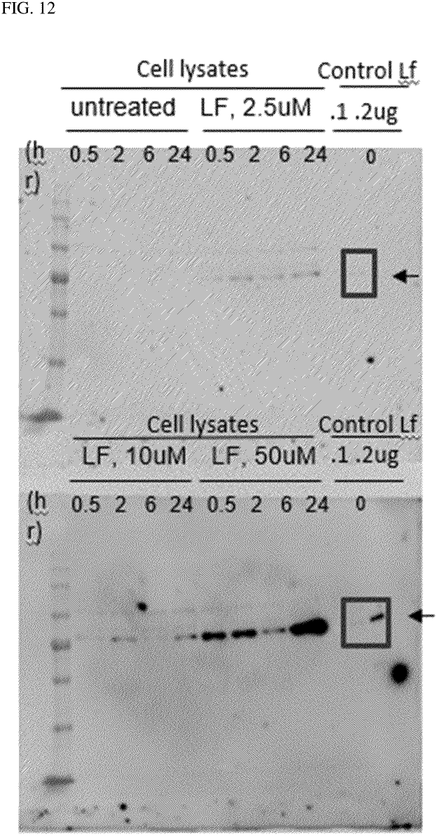

To show that hLF enters, accumulates, and remains intact in cells, VeroE6/T cells (5×10⁴ cells in a 24-well plate) were incubated with hLF at 2.5 µM, 10 µM, and 50 µM. Cells were harvested at 0.5, 2, 6, and 24, hours. Cells were then lysed, centrifuged, and the supernatant containing the cytoplasmic fraction was subjected to western blot analysis (FIG. 12). Samples were boiled in gel running buffer containing SDS and DTT and subjected to SDS-PAGE. As shown in FIG. 13, the control lanes (last two lanes) contained 100 ng and 200 ng of hLF, respectively. The hLF band was detected using anti-hLF antibody (sc-53498) at 1:1,000 dilution. LF found in cell lysate co-migrated with the untreated control LF.

What is claimed is:

1. A recombinant polypeptide comprising:
a) a lactoferrin protein or fragment thereof;
b) a linker; and
c) a SARS-CoV or SARS-CoV-2 Spike (S)-protein or fragment thereof;
wherein the lactoferrin protein or fragment thereof binds to heparan sulfate proteoglycans (HSPG) on a host cell surface,
wherein the S-protein or fragment thereof binds to the ACE2 receptor on the host cell surface, and
wherein the recombinant polypeptide inhibits binding of a virus to the host cell.

2. The recombinant polypeptide of claim 1, wherein the Spike protein comprises a sequence as set forth in SEQ ID NO:1-2.

3. The recombinant polypeptide of claim 1, wherein the lactoferrin protein comprises a sequence as set forth in SEQ ID NOs:3-4.

4. The recombinant polypeptide of claim 1, wherein the linker comprises a sequence as set forth in SEQ ID NOs:5-6.

5. The recombinant polypeptide of claim 1, wherein the recombinant polypeptide comprises a sequence set forth in SEQ ID NO:7-8.

6. The recombinant polypeptide of claim 1, further comprising an immunoglobulin (Ig) Fc-domain.

7. The recombinant polypeptide of claim 1, wherein the recombinant polypeptide comprises a sequence set forth in SEQ ID NO:9-10.

8. The recombinant polypeptide of claim 1, wherein the virus is a virus from the Coronaviridae family.

9. The recombinant polypeptide of claim 8, wherein the Coronaviridae virus is Severe Acute Respiratory Syndrome (SARS).

10. The recombinant polypeptide of claim 9, wherein the severe acute respiratory syndrome (SARS) virus is SARS-CoV or SARS-CoV-2.

11. A pharmaceutical composition comprising the recombinant polypeptide of claim 1.

12. A method of preventing or treating SARS-CoV-2 infection in a subject exposed to SARS-CoV-2, comprising administering to the subject a therapeutically or prophylactically effective amount of the pharmaceutical composition of claim 11.

13. The method of claim 11, wherein the pharmaceutical composition is administered via the oral, mucosal, nasopharyngeal, or parenteral routes.

14. The method of claim 13, further comprising a therapeutically effective amount of at least a second treatment.

15. The method of claim 14, wherein the second treatment comprises ivermectin, Remdesivir®, a monoclonal antibody, Regeneron®, hydroxychloroquine, Momupiravir, and/or Paxlovid.

16. A method of preventing or treating SARS-CoV-2 infection in a subject exposed to SARS-CoV-2, comprising administering to the subject:
(a) a prophylactically effective amount of a lactoferrin protein or fragment thereof; or
(b) a prophylactically effective amount of ivermectin; or
(c) a prophylactically effective amount of a lactoferrin protein or fragment thereof and a prophylactically effective amount of ivermectin; or
(d) a prophylactically effective amount of a recombinant lactoferrin protein or fragment thereof; or
(e) a prophylactically effective amount of a recombinant lactoferrin protein or fragment thereof and a prophylactically effective amount of ivermectin.

17. A method of treating or preventing an early stage SARS-CoV-2 infection comprising administering to the subject:
(a) a therapeutically effective amount of a lactoferrin protein or fragment thereof; or
(b) a therapeutically effective amount of a lactoferrin protein or fragment thereof and a therapeutically effective amount of ivermectin; or
(c) a therapeutically effective amount of a recombinant lactoferrin protein or fragment thereof; or
(d) a therapeutically effective amount of a recombinant lactoferrin protein or fragment thereof and a therapeutically effective amount of ivermectin.

18. A method of treating or preventing a late stage SARS-CoV-2 infection comprising administering to the subject:
(a) a therapeutically effective amount of a lactoferrin protein or fragment thereof; or
(b) a therapeutically effective amount of a lactoferrin protein or fragment thereof and a therapeutically effective amount of ivermectin; or
(c) a therapeutically effective amount of a recombinant lactoferrin protein or fragment thereof; or
(d) a therapeutically effective amount of a recombinant lactoferrin protein or fragment thereof and a therapeutically effective amount of ivermectin.

19. The method of any of claims 16-18, wherein the lactoferrin protein is human lactoferrin.

20. The method of claim 19, wherein the lactoferrin protein binds to the cell membrane of cells in a subject and is taken into the cell upon binding.

21. The method of claim 20, wherein the lactoferrin is present in the cytoplasm.


SEQUENCE LISTING (10 sequences)

SEQ ID NO: 1 - DNA, 216 bp, Severe acute respiratory syndrome-related coronavirus
  source 1..216: mol_type = unassigned DNA
aattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatac

SEQ ID NO: 2 - protein, 72 aa, Severe acute respiratory syndrome-related coronavirus
NSNNLDSKVGGNYNYLYRLFRKSNLKPFERDISTEIYQAGSTPCNGVEGFNCYFPLQSYGFQPTNGVGYQPY

SEQ ID NO: 3 - DNA, 2136 bp, Homo sapiens
  source 1..2136: mol_type = genomic DNA
atgaaacttgtcttcctcgtcctgctgttcctcggggccctcggactgtgtctggctggccgtaggagaaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatgaaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaa

SEQ ID NO: 4 - protein, 711 aa, Homo sapiens [sequence omitted]

SEQ ID NO: 5 - DNA, 12 bp, synthetic construct
  misc_feature 1..12: note = 4-glycine linker
  source 1..12: mol_type = other DNA
ggcggcggcggc

SEQ ID NO: 6 - protein, 4 aa, synthetic construct
  REGION 1..4: note = 4-glycine linker
GGGG

SEQ ID NO: 7 - DNA, 2817 bp, synthetic construct
  misc_feature 1..2817: note = DNA sequence of hLF-CoV2-RBD construct
  source 1..2817: mol_type = other DNA
atgaaacttgtcttcctcgtcctgctgttcctcggggccctcggactgtgtctggctggccgtaggagaaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatgaaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagggcggcggcggcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttctaa

SEQ ID NO: 8 - protein, 938 aa, synthetic construct
  REGION 1..938: note = Amino acid sequence of hLF-CoV2-RBD construct
MKLVFLVLLFLGALGLCLAGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDEGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKGGGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 9 - DNA, 3546 bp, synthetic construct
  misc_feature 1..3546: note = DNA sequence of LF-Fc-RBD construct
  source 1..3546: mol_type = other DNA
atgtacaggatgcaactcctgtcttgcattgcactaagtcttgcacttgtcacgaattcgggctcgaggggccgtaggagaaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatgaaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaaggggccctcgggctcgagtgctgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggcggcggcagggtccaaccaacagagagcattgtgaggtttccaaacatcaccaacctgtgtccatttggagaggtgttcaatgccaccaggtttgcctctgtctatgcctggaacaggaagaggattagcaactgtgtggctgactactctgtgctctacaactctgcctccttcagcaccttcaagtgttatggagtgagcccaaccaaactgaatgacctgtgtttcaccaatgtctatgctgactcctttgtgattaggggagatgaggtgagacagattgcccctggacaaacaggcaagattgctgactacaactacaaactgcctgatgacttcacaggctgtgtgattgcctggaacagcaacaacctggacagcaaggtgggaggcaactacaactacctctacagactgttcaggaagagcaacctgaaaccatttgagagggacatcagcacagagatttaccaggctggcagcacaccatgtaatggagtggagggcttcaactgttactttccactccaatcctatggcttccaaccaaccaatggagtgggctaccaaccatacagggtggtggtgctgtcctttgaactgctccatgcccctgccacagtgtgtggaccaaagaagagcaccaacctggtgaagaacaagtgtgtgaacttctag

SEQ ID NO: 10 - protein, 1181 aa, synthetic construct
  REGION 1..1181: note = Amino acid sequence of LF-Fc-RBD construct
MYRMQLLSCIALSLALVTNSGSRGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDEGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRKGPSGSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF